(19) United States
(12) Patent Application Publication
(10) Pub. No.: US 2019/0224317 A1
(43) Pub. Date: Jul. 25, 2019

(54) BIOPHOTONIC COMPOSITIONS AND METHODS FOR REDUCING SCARRING

(71) Applicant: KLOX TECHNOLOGIES INC., Laval (CA)

(72) Inventors: Remigio PIERGALLINI, San Benedetto del Tronto (IT); Nikolaos LOUPIS, Athens (GR); Andreas NIKOLIS, Ville Mont-Royal (CA); Stéphane FAUVERGHE, Montreal (CA); Lise HEBERT, Montreal (CA); Francesco BELLINI, Calgary (CA)

(21) Appl. No.: 16/333,707

(22) PCT Filed: Sep. 22, 2017
(86) PCT No.: PCT/CA2017/051118

§ 371 Date: Mar. 15, 2019

Related U.S. Application Data
(60) Provisional application No. 62/399,017, filed on Sep. 23, 2016.

(51) Int. Cl. A61K 41/00; A61K 9/00; A61P 17/02

(52) U.S. Cl. CPC A61K 41/0057; A61P 17/02; A61K 9/0014

(57) ABSTRACT

The present disclosure relates to methods for reducing scarring of wounds. The methods comprising topically applying on the wound a biophotonic composition followed by illumination of the applied biophotonic composition with actinic light, wherein the method comprises the following schedule: (a) a period of from about 1 day to about 4 weeks during which, at least once every week the biophotonic composition is topically applied onto the wound and is illuminated for a period of at least 5 minutes, followed by a rest period of less than about a week; and (b) repeating step (a) over a period of at least 4 weeks.

### CROSS-REFERENCE TO RELATED APPLICATION

This application claims the benefit of and priority to U.S. provisional patent application No. 62/399,017, filed on Sep. 23, 2016; the content of which is herein incorporated in entirety by reference.

### FIELD OF TECHNOLOGY

The present disclosure generally relates to biophotonic compositions, biophotonic methods and treatments for reducing scarring of wounds of a skin or a soft tissue. The present disclosure also generally relates to the use of biophotonic compositions for reducing scarring of wounds, and improve cosmesis. The present disclosure further generally relates to kits for reducing scarring of wounds.

### BACKGROUND INFORMATION

Mammals skin has the ability to heal itself in response to different insults. However, the healing process often leads to the formation of scars such as keloids and/or hypertrophic scars, which are typically abnormal responses to injury.

Scars are classified into different categories, based on the nature of the injury having caused the scar, its clinical characteristics and its appearance. Flat or pale scars are the most common type of scar and result from the body's natural healing process. Initially, these scars may be red or dark and raised (increased thickness) after the wound has healed but they eventually become paler and flatten naturally over time, resulting in a flat, pale scar. This process can take up to two years and there will always be some visible evidence of the original wound.

Hypertrophic scars are more common in young and people with darker skin. When a normal wound heals, the body produces new collagen fibres at a rate which balances the breakdown of old collagen. Hypertrophic scars are red and thick and may be itchy or painful. They do not extend beyond the boundary of the original wound but may continue to thicken for up to 6 months. They usually improve over the next one to two years but may cause distress due to their appearance or the intensity of the itching, also restricting movement (pliability) if they are located close to a joint.

Similarly to hypertrophic scars, keloids are the result of an imbalanced collagen production in a healing wound. Unlike hypertrophic scars, keloids grow beyond the boundary of the original wound and can continue to grow indefinitely. They may be itchy or painful and most will not improve in appearance over time. Keloid scars can result from any type of injury to the skin, including scratches, injections, insect bites and tattoos.

Sunken scars are recessed into the skin. They may be due to the skin being attached to deeper structures (such as muscles) or to loss of underlying fat. They are usually the result of an injury.

A very common cause of sunken scarring is acne or chicken pox which can result in a pitted appearance. However, acne scarring is not always sunken in appearance and can even become keloid.

Stretched scars occur when the skin around a healing wound is put under tension during the healing process. This type of scarring may follow injury or surgery. Initially, the scar may appear normal but can widen and thin over a period of weeks or months. This can occur where the skin is close to a joint and is stretched during movement or may be due to poor healing due to general ill health or malnutrition.

Three distinct phases are involved in the pathophysiology of excessive scar formation: inflammation, proliferation and remodelling. In normal wound healing, during the inflammation phase, platelets degranulation will be responsible for the release and activation of an array of different potent cytokines which will serve as chemotactic agents to recruit macrophages, neutrophils, epithelial cells and fibroblasts. In normal conditions, a balance will be achieved between new tissue biosynthesis and degradation mediated by apoptosis and remodeling of the extracellular matrix. In excessive scarring, a persistent inflammation, caused by an increased secretion of different factors (TGF-β1, TGF-β2, PDGF, IGF-1, IL-4 and IL-10) might lead to an excessive collagen synthesis or deficient matrix degradation and remodeling.

Different tools are available, to be used either by the clinician or even by the patient him/herself to evaluate scars. The Patient and Observer Scar Assessment Scale (POSAS) is designed to be used by both the clinician and the patient. The clinician will assess the scar looking at vascularity, pigmentation, thickness, relief, pliability and importance of surface area whereas the patient will look after pain, itching, color, stiffness, thickness, contour irregularities and overall opinion. The Vancouver Scar Scale (VSS) is another validated scale used for scars assessment.

Reduction of scarring represents a significant and largely unmet medical need in a wide variety of clinical settings. Interestingly, patients will appreciate even minimal improvements in scar appearance and most of them are more sensitive to scarring than many clinicians.

To this day, silicone sheeting (silicone sheets) and other applications such as vitamin E and massage, are considered as the first-line prophylactic and treatment option for reducing scarring. Although the application of silicone sheeting has shown that the appearance of the scar has improved, silicone sheeting does not easily adapt to the topography of the scar. For example, silicone sheeting applied onto a scar with an uneven topography would not reach derepressed portions of the scar. In addition, silicone sheetings typically have to be worn over an extended period of time creating discomfort to the user.

As such, there remains a need in the art for methods and treatments of reducing scarring of wounds that are more easily adaptable to the topography of a scar and/or wound to be reduced. There also remains a need in the art for faster and more efficient methods and treatments for reducing scarring that do not cause discomfort to the subject.

### SUMMARY OF DISCLOSURE

In one aspect, the present disclosure relates to methods that may be used for reduction of scarring of wounds, in particular for reduction of scarring of wounds on the skin or wounds on a soft tissue.

In some aspects, the present disclosure relates to a method for reducing scarring of an acute wound. In some other aspects, the present disclosure relates to a method for reducing scarring of chronic wounds.

In some aspects, the present disclosure relates to a method for reducing scarring of a wound that comprises topically applying a biophotonic composition to the wound and illuminating the applied biophotonic composition for a time sufficient to activate the biophotonic composition; wherein the steps of applying the biophotonic composition and the step of illuminating the biophotonic composition are performed at least once weekly.

In some other aspects, the present disclosure relates to a method for reducing scarring of a wound, wherein the method comprises topically applying a biophotonic composition to the wound and illuminating the applied biophotonic composition for a time sufficient to activate the biophotonic composition; wherein the steps of applying the biophotonic composition and the step of illuminating the biophotonic composition are performed at least twice weekly.

In some other aspects, the present disclosure relates to a method for reducing scarring of a wound, wherein the method comprises topically applying a biophotonic composition to the wound, and illuminating the applied biophotonic composition for a time sufficient to activate the biophotonic composition; wherein the steps of applying the biophotonic composition and the step of illuminating the biophotonic composition are performed consecutively once weekly.

As used herein, the expression “consecutively once weekly” indicates that the second occurrence of the method of the present disclosure is performed right after the first occurrence of the method (e.g., the first and second occurrences of the method of the present disclosure are performed back to back). In such instances, the biophotonic composition is applied, illuminated and then washed off the wound. Then a fresh amount of biophotonic composition is applied right away and illuminated.

In some aspects, the present disclosure relates to a method for reducing scarring of a wound, wherein the method comprises topically applying on the wound a biophotonic composition followed by illumination of the applied biophotonic composition with actinic light, wherein the method comprises the following schedule: (a) at least once weekly: i) topically applying a first amount of biophotonic composition on the wound, illuminating the applied first amount of biophotonic composition for a period of at least 5 minutes, removing the applied first amount of biophotonic composition from the wound; and ii) topically applying a second amount of biophotonic composition on the wound, illuminating the applied second amount of biophotonic composition for a period of at least 5 minutes, removing the applied second amount of biophotonic composition from the wound; and iii) initiating a rest period of less than about a week; and (b) repeating step (a) over a period of at least 4 weeks.

In some aspects, the present disclosure relates to a method for reducing scarring of a wound, the method comprising: a) at least once weekly, performing an application step followed by an illumination step, wherein the application step comprises topically applying onto the wound a biophotonic composition and wherein the illumination step comprises illuminating the applied biophotonic composition with actinic light for at least 5 minutes; b) allowing a rest period of less than about one week between the illumination step and a subsequent application step; and c) repeating a) and b) over a period of at least about 4 weeks.

In certain embodiments, the biophotonic composition may be applied onto the wound at least once weekly, at least twice weekly, at least three times weekly, at least four times weekly, at least five times weekly, or at any other frequency that may be suitable.

In certain embodiments, a rest period is introduced between occurrences of the methods defined herein being performed. The rest period (holiday period) can be for less than about 7 days, or less than about 6 days, or less than about 5 days, or less than about 4 days, or less than about 3 days, or less than about 2 days, or less than about a week.

In some aspects, the method as defined herein is performed over a period of at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least 17 weeks, at least about 18 weeks, at least about 19 weeks, at least about 20 weeks, at least about 21 weeks, at least about 22 weeks, at least about 23 weeks, at least about 24 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks or at least about 52 weeks, or for any other length of time deemed appropriate.

In some aspects, the method as defined herein is performed over a period that is less than about 24 weeks, less than about 23 weeks, less than about 22 weeks, less than about 21 weeks, less than about 20 weeks, less than about 19 weeks, less than about 18 weeks, less than about 17 weeks, less than about 16 weeks, less than about 15 weeks, less than about 14 weeks, less than about 13 weeks, less than about 12 weeks, less than about 11 weeks, less than about 10 weeks, less than about 9 weeks, less than about 8 weeks, less than about 7 weeks, less than about 6 weeks, less than about 5 weeks, or less than about 4 weeks.

In some instances, the method defined herein further comprises a step of removing the used biophotonic composition following illumination of the biophotonic composition. As used herein the expression “used biophotonic composition” refers to a biophotonic composition that has been illuminated. As used herein the expression “fresh biophotonic composition” refers to a biophotonic composition that has not been illuminated.

Removal of the used biophotonic composition may be carried out by wiping the used biophotonic composition off the wound with, for example, a cloth and/or washing the used biophotonic composition off the wound with, for example, a liquid.

In some aspects, the method defined herein comprises illuminating the biophotonic composition applied to the wound. Illumination of the biophotonic composition may be performed for a period which can be up to about 5 minutes, up to about 6 minutes, up to about 7 minutes, up to about 8 minutes, up to about 9 minutes, up to about 10 minutes, up to about 15 minutes, up to about 20 minutes, up to about 25 minutes, or up to about 30 minutes. The illumination time may comprise the total length of time that the biophotonic composition is in contact with the wound.

In some aspects, the methods of the present disclosure improve vascularity of the wounds. Improvement of vascularization of wounds includes promoting and/or increasing vascularization of the wound.

In some aspects, the methods of the present disclosure improve pigmentation, and lowers hyperpigmentation, of the wounds. Improvement of pigmentation of wounds includes partial or substantial restoration of original skin/soft tissue pigmentation (i.e., pigmentation of the skin/soft tissue prior to occurrence of the wound).

In some aspects, the methods of the present disclosure reduce thickness of the wounds so as to restore the overall thickness of the wounds to the original thickness of the skin and/or soft tissues (i.e., thickness of the skin/soft tissue prior to occurrence of the wound).

In some aspects, the methods of the present disclosure reduce the surface area of the wounds, reduce itchiness associated with the wounds, reduce stiffness of the wounds, and/or increase pliability of the wounds.

In certain embodiments, the biophotonic composition useful in the methods of the present disclosure is a topical biophotonic composition. In some instances, the biophotonic composition is a gel, a semi-solid or a viscous liquid, which can be spread in and/or onto the wound. In some embodiments, the biophotonic composition can remain on the wound when the wound is inverted or tilted. In some instances, the biophotonic composition may be applied in and/or onto the wound as well as onto a portion of the skin/tissue that surrounds the wound.

In a yet further aspect, the biophotonic composition useful in the methods of the present disclosure comprises at least one light-accepting molecule. In some instances, the at least one light-accepting molecule absorbs or emits light at a wavelength of between about 200 nm and about 600 nm, between about 400 nm and about 800 nm, or between about 400 nm and about 600 nm. In some instances, the at least one light-accepting molecule absorbs and/or emits light at a wavelength in the range of the visible spectrum. In some instances, the at least one light-accepting molecule can be a xanthene dye. The at least one light-accepting molecule can be Eosin Y, Eosin B, Erythrosin B, Fluorescein, Rose Bengal or Phloxin B. In some instances, the at least one light-accepting molecule is Eosin Y.

The at least one fluorescent light-accepting molecule can be present in the biophotonic composition in an amount of between about 0.001% and about 40% by weight of the total biophotonic composition, preferably between about 0.005% and about 2% by weight of the total biophotonic composition, more preferably between about 0.01% and about 2% by weight of the total biophotonic composition.

In some instances, the biophotonic composition useful in the methods of the present disclosure is as defined in WO 2011/134087 or as defined in WO 2015/000058, which are both incorporated herein by reference.

In certain embodiments, the methods of the present disclosure comprise illuminating the biophotonic composition applied onto the wounds is illuminated for an illumination period that is between about 5 minutes to about 30 minutes. In some instances, the illumination period is less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, less than about 11 minutes, less than about 10 minutes, less than about 9 minutes, less than about 8 minutes, less than about 7 minutes, or less than about 6 minutes.

In certain other embodiments, the methods of the present disclosure comprise illuminating the biophotonic composition applied onto the wounds where the wound is illuminated for an illumination period that is less than about 5 minutes, less than about 4 minutes, less than about 3 minutes, less than about 2 minutes, less than about 1 minute, less than about 30 seconds, less than about 20 seconds, or less than about 10 seconds. In some instances, the illumination periods is between about 10 seconds and about 60 seconds.

The illumination period can correspond to, or be longer than a time it takes for the at least one light-accepting molecule to photobleach.

In certain embodiments, the method of the present disclosure comprises illuminating the biophotonic composition for a period of at least 5 minutes, at least 6 minutes, at least 7 minutes, at least 10 minutes, at least 11 minutes, at least 12 minutes, at least 13 minutes, at least 14 minutes, at least 15 minutes, at least 20 minutes, at least 25 minutes, or at least 30 minutes.

The biophotonic composition may be illuminated with visible non-coherent light, such as violet and/or blue light. Any other suitable light source can be used. Preferably, the biophotonic composition is illuminated with a light having a wavelength that overlaps with an absorption spectrum of the at least one first light-accepting molecule.

The distance of the light source from the biophotonic composition may be any distance which can deliver an appropriate light power density to the biophotonic composition and/or to the wound, for example about 5 cm, about 6 cm, about 7 cm, about 8 cm, about 9 cm, about 10 cm, about 15 cm or about 20 cm. The biophotonic composition is applied topically at any suitable thickness. Typically, the biophotonic composition is applied topically in and/or onto the wound (including or not any surrounding areas) at a thickness of at least about 2 mm, about 2 mm to about 10 mm.

In certain aspects, the biophotonic composition is removed from the wound following the illumination step. Accordingly, the biophotonic composition is removed from the wound within at least 5 minutes, at least 6 minutes, at least 7 minutes, at least 8 minutes, at least 9 minutes, at least 10 minutes, at least 15 minutes, at least 20 minutes, at least 25 minutes or at least 30 minutes after application.

According to further aspects, the present disclosure relates to kits comprising a biophotonic composition as described herein, and one or more of a light source for activating the biophotonic composition, instructions for use of the biophotonic composition and/or the light source, and a device for applying and/or removing the biophotonic composition from the wound.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure and are not mutually exclusive can be combined with one another. Elements of one embodiment can be utilized in the other embodiments without further mention. A detailed description of specific embodiments is provided herein below with reference to the accompanying drawings in which:

FIGS. 1A-1G are graphs showing the effects on a POSAS Observer Scale of a biophotonic treatment according to one embodiment of the present disclosure compared to a treatment with silicone sheets, treatments were performed once weekly with the first occurrence being on day 7 post surgery. Graphs expressing mean change from baseline for Groups A1+B1. FIG. 1A shows overall opinion; FIG. 1B shows the total score; FIG. 1C shows the effects of the treatments on vascularization of the wound; FIG. 1D shows the effect of the treatments on pigmentation of the wound; FIG. 1E shows the effect of the treatments on thickness of the wound; FIG. 1F shows the effect of the treatments on pliability of the wound; FIG. 1G shows the effect of the treatments on surface area of the wound; and FIG. 1H shows the effect of the treatments on the relief experienced by the subject. “BT” refers to the biophotonic treatment according to one embodiment of the present disclosure.

FIGS. 2A-2G are graphs showing the effects on a POSAS Observer Scale of a biophotonic treatment according to one embodiment of the present disclosure compared to a treatment with silicone sheets, treatments were performed once weekly with the first occurrence being on day 21 post surgery. Graphs expressing mean change from baseline for Groups A2+B2. FIG. 2A shows overall opinion; FIG. 2B shows the total score; FIG. 2C shows the effect of the treatments on vascularization of the wound; FIG. 2D shows the effect of the treatment on pigmentation of the wound; FIG. 2E shows the effect of the treatments on thickness of the wound; FIG. 2F shows the effect of the treatments on pliability of the wound; FIG. 2G shows the effect of the treatments on surface area of the wound; and FIG. 2H shows the effect of the treatments on the relief experienced by the subject. “BT” refers to the biophotonic treatment according to one embodiment of the present disclosure.

FIGS. 3A-3G are graphs showing the effects on a POSAS Observer Scale of a biophotonic treatment according to one embodiment of the present disclosure compared to a treatment with silicone sheets, treatments were performed twice consecutively once weekly with the occurrence being on day 7 post surgery. Graphs expressing mean change from baseline for Groups C1+C2. FIG. 3A shows the overall opinion; FIG. 3B shows the total score; FIG. 3C shows the effect of the treatment on vascularization of the wound; FIG. 3D shows the effect of the treatments on pigmentation of the wound; FIG. 3E shows the effect of the treatments on thickness of the wound; FIG. 3F shows the effect of the treatments on pliability of the wound; FIG. 3G shows the effect of the treatments on surface area of the wound; and FIG. 3H shows the effect of the treatments on the relief experienced by the subject. “BT” refers to the biophotonic treatment according to one embodiment of the present disclosure.

FIGS. 4A-4D are graphs showing the effects on POSAS Scales of a biophotonic treatment according to one embodiment of the present disclosure compared to a treatment with silicone sheets. FIG. 4A shows the Total Score on a POSAS Observers Scale (mean change from baseline) for all comparison groups (Groups A1+A2+B1+B2+C1+C2). FIG. 4B shows the Total Score on a POSAS Patient Scale (mean change from baseline) for all comparison groups (Groups A1+A2+B1+B2+C1+C2). FIG. 4C shows the Overall Opinion on a POSAS Observers Scale (mean change from baseline) for all comparison groups (Groups A1+A2+B1+B2+C1+C2); and FIG. 4D shows the Overall Opinion on a POSAS Patient Scale (mean change from baseline) for all comparison groups (Groups A1+A2+B1+B2+C1+C2). FIGS. 5A-C are photographs of breasts from a subject that underwent breast reduction. The subject's right breast was treated with the method according to one embodiment of the present disclosure (left panel) wherein the method is performed twice consecutively once weekly, whereas the subject's left breast was treated with silicone sheets (right panel). FIG. 5A shows the breasts at week 7 post surgery; FIG. 5B shows the breasts at week 12 post surgery; FIG. 5C shows the breasts at week 24 post surgery. “BT” indicates the biophotonic treatment according to one embodiment of the present disclosure. “Sheets” refers to silicone sheets.

### DETAILED DISCLOSURE OF EMBODIMENTS

In some embodiments, the present disclosure relates to methods for reducing scarring of wounds of a skin and/or wounds of a soft tissue. Biophotonic compositions which are useful in the methods defined herein comprise at least one light-accepting molecule which may emit a therapeutic light or may promote a therapeutic effect on a wound by activating other components of the biophotonic composition. In some instances, the light-accepting molecules are exogenous (i.e., light-accepting molecules that are not naturally present in skin or tissue onto which the biophotonic composition as defined herein is to be applied).

In some embodiments, the biophotonic composition useful in the methods of the present disclosure is applied in and/or onto the wound. Preferably, the consistency of the biophotonic composition allows it reach derepressed portions that may be present in the wounds. In this manner, some of the beneficial effects of the biophotonic composition may be achieved on the surface of the wound as well as in the derepressed portions of the wound.

In some instances, the wound is a wound that has been closed with sutures. In some other instances, the wound is an acute wound. In some other instances, the acute wound is that from a surgical procedure. In some other instances, the wound is a chronic wound, such as a venous leg ulcer or a pressure ulcer.

Before continuing to describe the present disclosure in further detail, it is to be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Biophotonic” means the generation, manipulation, detection and application of photons in a biologically relevant context. In other words, biophotonic compositions exert their physiological effects primarily due to the generation and manipulation of photons, for example, by absorbing photons to emit photons or to transfer energy, for example, by absorbing photons to emit photons or to transfer energy.

As used herein the term “wound” means an injury to any tissue, including for example, acute, subacute, and non-healing wounds. Examples of wounds may include both open and closed wounds. Wounds include, for example, skin diseases that result in a break of the skin or in a wound, clinically infected wounds, burns, incisions, excisions, lesions, lacerations, abrasions, puncture or penetrating wounds, gunshot wounds, surgical wounds, contusions, hematomas, crushing injuries, ulcers, and scarring (cosmesis).

As used herein, the expression “non-healing wounds” means wounds that do not heal in an orderly set of stages and a predictable amount of time and rate in the way that most normally-healing wounds heal, and non-healing wounds include, but are not limited to: incompletely healed wounds, delayed healing wounds, impaired wounds, difficult to heal wounds and chronic wounds. Examples of such non-healing wounds include diabetic foot ulcers, vascultic ulcers, pressure ulcers, decubitus ulcers, infectious ulcers, trauma-induced ulcers, burn ulcers, ulcerations associated with pyoderma gangrenosum, dehiscent and mixed ulcers. A non-healing wound may include, for example, a wound that is characterized at least in part by: 1) a prolonged inflammatory phase, 2) a slow forming extracellular matrix, and/or 3) a decreased rate of epithelialization or closure.

As used herein, the expression “chronic wound” means a wound that has not healed within about 4 to 6 weeks. Chronic wounds include venous ulcers, venous stasis ulcers, arterial ulcers, pressure ulcers, diabeteic ulcers, and diabetic foot ulcers.

As used herein the expression “acute wounds” includes injuries to the skin or to soft tissues that occur suddenly rather than over time. Acute wounds include surgical wounds which themselves include incisions made purposefully by, for example, a health care professional and are cut precisely, creating clean edges around the wound. Surgical wounds may be closed or left open to heal. Acute wounds also include traumatic wounds which include injuries to the skin and underlying tissue caused by a force of some nature.

As used herein the expression “wound that has been closed” refers to a wound that has been closed by a wound closure technique with, for example, suture materials, staples, tapes, adhesive compounds, grafts, skin/soft tissue transplants, or the like.

As used herein, the expression “soft tissue” includes tendons, ligaments, fascia, fibrous tissues, fat, synovial membranes, muscles, nerves and blood vessels.

“Gels” are defined as substantially dilute cross-linked systems. Gels may be semi-solids and exhibit substantially no flow when in the steady state at room temperature (e.g. about 20° C.-25° C.). By steady state is meant herein during a treatment time and under treatment conditions. Gels, as defined herein, may be physically or chemically cross-linked. As defined herein, gels also include gel-like compositions such as viscous liquids.

“Topical” means as applied to body surfaces, such as the skin, mucous membranes, vagina, oral cavity, soft tissues, internal surgical wound sites, and the like.

The terms and expressions light-accepting molecule“photoactivating agent”, “photoactivator”, and “chromophore” are used herein interchangeably. A light-accepting molecule means a chemical compound, when contacted by light irradiation, is capable of absorbing the light. The light-accepting molecule readily undergoes photoexcitation and can then transfer its energy to other molecules or emit it as light.

“Photobleaching” means the photochemical destruction of a light-accepting molecule.

The expression “actinic light” is intended to mean light energy emitted from a specific light source (e.g., lamp, LED, or laser) and capable of being absorbed by matter (e.g. the light-accepting molecule or photoactivator defined above). The expression “actinic light” and the term “light” are used herein interchangeably. In a preferred embodiment, the actinic light is visible light.

As used herein, a “hygroscopic” substance is a substance capable of taking up water, for example, by absorption or adsorption even at relative humidity as low as 50%, at room temperature (e.g., about 20° C.−25° C.).

Biophotonic compositions are compositions that are activated by light (e.g., photons) of specific wavelength. Biophotonic compositions comprise at least one light-accepting molecule which is activated by light and accelerates the dispersion of light energy, which leads to light carrying on a therapeutic effect on its own, and/or to the photochemical activation of other agents that may be present in the biophotonic composition (e.g., acceleration in the breakdown process of peroxide, which is an oxygen-releasing agent) when such compound is present in the biophotonic composition or at the treatment site, leading to the formation of oxygen radicals, such as singlet oxygen. The biophotonic composition may comprise an oxygen-releasing agent which, when mixed with the first light-accepting molecule and subsequently activated by light, can be photochemically activated which may lead to the formation of oxygen radicals, such as singlet oxygen.

In some aspects, the biophotonic compositions useful in the methods of the present disclosure comprise at least a first light-accepting molecule in a medium, wherein the composition is substantially resistant to leaching such that a low or negligible amount of the light-accepting molecule leaches out of the biophotonic composition into for example skin or onto a soft tissue onto which the biophotonic composition is applied. In certain embodiments, this is achieved by the medium comprising a gelling agent which slows or restricts movement or leaching of the light-accepting molecule.

In some aspects, the biophotonic compositions useful in the methods of the present disclosure do not stain the tissue onto which they are topically applied. Staining is determined by visually assessing whether the biophotonic composition colorizes white test paper saturated with 70% by volume ethanol/30% by volume water solution placed in contact with the biophotonic composition for a period of time corresponding to a desired illumination time. In some embodiments, a biophotonic composition of the present disclosure does not visually colorize white test paper saturated with a 70% by volume ethanol/30% by volume water solution placed in contact with the biophotonic composition under atmospheric pressure for a time corresponding to a desired illumination time.

In some instances, the biophotonic compositions are substantially transparent or translucent, or both, and/or have high light transmittance in order to permit light dissipation into and through the biophotonic composition. In this way, the area of tissue under the biophotonic composition can be treated both with the fluorescent light emitted by the biophotonic composition and the light irradiating the biophotonic composition to activate it, which may benefit from the different therapeutic effects of light having different wavelengths.

The % transmittance of the biophotonic composition can be measured in the range of wavelengths from 250 nm to 800 nm using, for example, a Perkin-Elmer Lambda 9500 series UV-visible spectrophotometer. Alternatively, a Synergy HT spectrophotometer (BioTek Instrument, Inc.) can be used in the range of wavelengths from 380 nm to 900 nm.

Transmittance is calculated according to the following equation:

$${A}_{λ} = {log}_{10} \frac{{I}_{0}}{I} = {log}_{10} \frac{1}{T} .$$

where A is absorbance, T is transmittance, I₀ is intensity of radiation before passing through material, I is intensity of light passing through material.

The values can be normalized for thickness. As stated herein, % transmittance (translucency) is as measured for a 2 mm thick sample at a wavelength of 526 nm. It will be clear that other wavelengths can be used.

In some embodiments, the biophotonic composition has a transparency or translucency that exceeds 15%, 20%, 25%, 30%, 35%, 40%, 45%, 50%, 55%, 60%, 65%, 70%, 75%, 80%, or 85%. In some embodiments, the transparency exceeds 70%, 80%, 85%, 86%, 87%, 88%, 89%, 90%, 91%, 92%, 93%, 94%, 95%, 96%, 97%, 98% or 99%. All transmittance values reported herein are as measured on a 2 mm thick sample using the Synergy HT spectrophotometer at a wavelength of 526 nm.

Embodiments of the methods of the present disclosure are for topical uses. For such uses, the biophotonic composition can be in the form of a semi-solid or viscous liquid, having properties such that less than 15% by weight of the total light-accepting molecule amount leaches out of the biophotonic composition in use. Preferably, the biophotonic compositions are a gel or are gel-like, including viscous liquids, and which have a spreadable consistency at room temperature (e.g. about 20° C.−25° C.), prior to illumination. By spreadable is meant that the biophotonic composition can be topically applied to the wound at a thickness of about 2 mm. Spreadable biophotonic compositions can conform to topography of the wound. This can have advantages over a non-conforming material in that a better and/or more complete illumination of the wound can be achieved.

In some embodiments, the biophotonic compositions useful in the methods of the present disclosure comprise at least one light-accepting molecule. The light-accepting molecules are contained or held within the biophotonic composition such that they do not substantially contact the target tissue to which the biophotonic composition is applied. In this way, the beneficial and therapeutic properties of the light-accepting molecule can be harnessed without the possibly damaging effects caused by light-accepting molecule-to-cell contact.

Suitable light-accepting molecules can be fluorescent dyes (or stains), although other dye groups or dyes (biological and histological dyes, food colorings, carotenoids, and other dyes) can also be used. Suitable light-accepting molecules can be those that are Generally Regarded As Safe (GRAS), although light-accepting molecules which are not well tolerated by the skin or other tissues can be included in the biophotonic composition as contact with the skin is minimal in use due to the leaching-resistant nature of the biophotonic composition.

In certain embodiments, the biophotonic composition comprises at least one light-accepting molecule which undergoes partial or complete photobleaching upon application of light. In some embodiments, the at least one light-accepting molecule absorbs and/or emits at a wavelength in the range of the visible spectrum, such as at a wavelength of between about 380-800 nm, between about 380-700 nm, or between about 380-600 nm. In other embodiments, the at least one light-accepting molecule absorbs/or emits at a wavelength of between about 200-800 nm, between about 200-700 nm, between about 200-600 nm or between about 200-500 nm. In other embodiments, the at least one light-accepting molecule absorbs/or emits at a wavelength of between about 200-600 nm. In some embodiments, the at least one light-accepting molecule absorbs/or emits light at a wavelength of between about 200-300 nm, between about 250-350 nm, between about 300-400 nm, between about 350-450 nm, between about 400-500 nm, between about 400-600 nm, between about 450-650 nm, between about 600-700 nm, between about 650-750 nm or between about 700-800 nm.

It will be appreciated to those skilled in the art that optical properties of a particular light-accepting molecule may vary depending on the light-accepting molecule's surrounding medium. Therefore, as used herein, a particular light-accepting molecule's absorption and/or emission wavelength (or spectrum) corresponds to the wavelengths (or spectrum) measured in a biophotonic composition useful in the methods of the present disclosure.

In certain embodiments, the biophotonic topical composition of the present disclosure further comprises a second light-accepting molecule. In some embodiments, the first light-accepting molecule has an emission spectrum that overlaps at least about 80%, 75%, 70%, 65%, 60%, 55%, 50%, 45%, 40%, 35%, 30%, 25%, 20%, 15%, or at least about 10% with an absorption spectrum of the second light-accepting molecule. In one embodiment, the first light-accepting molecule has an emission spectrum that overlaps at least about 20% with an absorption spectrum of the second light-accepting molecule. In some embodiments, the first light-accepting molecule has an emission spectrum that overlaps at least 1-10%, 5-15%, 10-20%, 15-25%, 20-30%, 25-35%, 30-40%, 35-45%, 50-60%, 55-65%, 60-70%, 65-75%, 70-80%, 75-80% with an absorption spectrum of the second light-accepting molecule.

% spectral overlap, as used herein, means the % overlap of a donor light-accepting molecule's emission wavelength range with an acceptor light-accepting molecule's absorption wavelength range, measured at spectral full width quarter maximum (FWQM). The spectral FWQM of the acceptor light-accepting molecule's absorption spectrum is from about 60 nm (515 nm to about 575 nm). The overlap of the donor light-accepting molecule's spectrum with the absorption spectrum of the acceptor light-accepting molecule is about 40 nm (from 515 nm to about 555 nm). Thus, the % overlap can be calculated as 40 nm/60 nm×100=66.6%.

In some embodiments, the second light-accepting molecule absorbs at a wavelength in the range of the visible spectrum. In certain embodiments, the second light-accepting molecule has an absorption wavelength that is relatively longer than that of the first light-accepting molecule within the range of about 50-250 nm, 25-150 nm or 10-100 nm.

In some embodiments, the light-accepting molecule or light-accepting molecules are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between 0.005 to about 10 mW/cm², about 0.5 to about 5 mW/cm².

Suitable light-accepting molecules that may be used in the biophotonic topical compositions useful in the methods of the present disclosure include, but are not limited to the following: chlorophyll dyes, xanthene dyes, methylene blue dyes, azo dyes.

Examples of chlorophyll dyes, include but are not limited to, chlorophyll a; chlorophyll b; oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Examples of xanthene dyes, include but are not limited to, eosin B; eosin B (4′,5′-dibromo,2′,7′-dinitr-o-fluorescein, dianion); eosin Y; eosin Y (2′,4′,5′,7′-tetrabromo-fluoresc-ein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′5-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin; eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Examples of azo dyes, include but are not limited to, methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In certain embodiments, the biophotonic composition useful in the methods of the present disclosure includes any of the light-accepting molecules listed above, or a combination thereof, so as to provide a biophotonic impact at the site of the wound. This is a distinct application of these agents and differs from the use of light-accepting molecules as simple stains or as a catalyst for photo-polymerization.

Light-accepting molecules can be selected, for example, on their emission wavelength properties in the case of fluorophores, on the basis of their energy transfer potential, their ability to generate reactive oxygen species, or their antimicrobial effect. These needs may vary depending on the condition requiring treatment. For example, chlorophylls may have an antimicrobial effect on bacteria.

In some embodiments, the biophotonic composition includes Eosin Y as the at least one light-accepting molecule. In the embodiments, where the biophotonic composition comprises at least two light-accepting molecules, the first light-accepting molecule may be Eosin Y and the second light-accepting molecule any be one or more of Rose Bengal, Erythrosin, and Phloxine B. It is believed that these combinations have a synergistic effect as Eosin Y can transfer energy to Rose Bengal, Erythrosin or Phloxine B when activated. This transferred energy is then emitted as fluorescence or by production of reactive oxygen species. This absorbed and re-emitted light is thought to be transmitted throughout the composition, and also to be transmitted into the site of treatment.

In further implementations of this embodiment, the biophotonic composition includes the following synergistic combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine. Other synergistic light-accepting molecule combinations are also possible.

In some embodiments, the biophotonic compositions useful in the methods of the present disclosure may include one or more gelling agents.

The present disclosure provides biophotonic compositions that comprise at least a first light-accepting molecule and a gelling agent, wherein the gelling agent provides a barrier such that the at least one light-accepting molecule of the biophotonic compositions are substantially not in contact with the target tissue. The gelling agent, when present in the biophotonic compositions, can render the biophotonic compositions substantially resistant to leaching such that the light-accepting molecule(s) or photosensitive agent(s) of the biophotonic topical compositions are not in substantial contact with the target tissue.

In certain embodiments, the biophotonic composition allows less than 30%, 25%, 20%, 15%, 10%, 9%, 8%, 7%, 6%, 5%, 4%, 3%, 2%, 1%, 0.8%, 0.5% or 0.1%, or essentially none of said light-accepting molecule content to leach out of the biophotonic composition.

In some embodiments, the biophotonic composition limits leaching of the at least one light-accepting molecule such that less than 15% by weight of the total light-accepting molecule amount leaches out of the biophotonic composition in use is topically applied onto tissue and illuminated with light. In some embodiments, the biophotonic composition limits leaching of the at least one light-accepting molecule such that less than 30%, 25%, 20%, 15%, 10%, 9%, 8%, 7%, 6%, 5%, 4%, 3%, 2%, 1%, 0.8%, 0.5% or 0.1% or essentially 0% of total light-accepting molecule amount can leach out into tissue during a treatment time in which the composition is topically applied onto tissue and illuminated with light.

A gelling agent for use according to the present disclosure may comprise any ingredient suitable for use in a topical biophotonic composition as described herein. The gelling agent may be an agent capable of forming a cross-linked matrix, including physical and/or chemical cross-links. The gelling agent is preferably biocompatible, and may be biodegradable. In some embodiments, the gelling agent is able to form a hydrogel or a hydrocolloid. An appropriate gelling agent is one that can form a viscous liquid or a semisolid. In preferred embodiments, the gelling agent and/or the biophotonic composition has appropriate light transmission properties. The gelling agent preferably allows biophotonic activity of the light-accepting molecule(s). For example, some light-accepting molecules require a hydrated environment in order to fluoresce. The gelling agent may be able to form a gel by itself or in combination with other ingredients such as water or another gelling agent, or when applied to a treatment site, or when illuminated with light.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); and polyvinylamines.

In some embodiments, the biophotonic compositions useful in the methods of the present disclosure comprise an oxygen-releasing agent. An example of oxygen-releasing agent is hydrogen peroxide (H₂O₂). Hydrogen peroxide for use in this biophotonic composition can be used in a gel, for example with 6% hydrogen peroxide. A suitable range of concentration over which hydrogen peroxide can be used in the present biophotonic composition is from about 0.1% to about 6%.

Another example of oxygen-releasing agent is urea hydrogen peroxide (also known as urea peroxide, carbamide peroxide or percarbamide) is soluble in water and contains approximately 35% hydrogen peroxide. Carbamide peroxide for use in the biophotonic composition can be used as a gel, for example with 16% carbamide peroxide that represents 5.6% hydrogen peroxide, or 12% carbamide peroxide. A suitable range of concentration over which urea peroxide can be used in the present biophotonic composition is from about 0.3% to about 16%.

Another example of oxygen-releasing agent is benzoyl peroxide which consists of two benzoyl groups (benzoic acid with the H of the carboxylic acid removed) joined by a peroxide group. A suitable range of concentration over which benzoyl peroxide can be used in the present composition is from about 2.5% to about 5%.

Peroxy acid, alkali metal peroxides, alkali metal percarbonates, peroxyacetic acid, and alkali metal perborates can also be included as the oxygen-releasing agent. Oxygen-releasing agents can be provided in powder, liquid or gel form.

In the biophotonic compositions and methods of the present disclosure, additional components may optionally be included, or used in combination with the biophotonic compositions as described herein. Such additional components include, but are not limited to, healing factors, growth factors, antimicrobials, wrinkle fillers (e.g. botox, hyaluronic acid or polylactic acid), collagens, anti-virals, anti-fungals, antibiotics, drugs, and/or agents that promote collagen synthesis. These additional components may be applied to the wound, skin or mucosa in a topical fashion, prior to, at the same time of, and/or after topical application of the biophotonic composition of the present disclosure, and may also be systemically administered. Suitable healing factors, antimicrobials, collagens, and/or agents that promote collagen synthesis are discussed below:

Healing factors comprise compounds that promote or enhance the healing or regenerative process of the tissues on the application site of the composition. During the photoactivation of the composition of the present disclosure, there may be an increase of the absorption of molecules at the treatment site by the skin, wound or the mucosa. An augmentation in the blood flow at the site of treatment is observed for a period of time. An increase in the lymphatic drainage and a possible change in the osmotic equilibrium due to the dynamic interaction of the free radical cascades can be enhanced or even fortified with the inclusion of healing factors. Suitable healing factors include, but are not limited to: hyaluronic acid, glucosamine, allantoin, saffron.

Examples of antimicrobials (or antimicrobial agent) are recited in U.S. Patent Application Publication Nos: 2004/0009227 and 2011/0081530, which are both herein incorporated by reference. Suitable antimicrobials for use in the methods of the present disclosure include, but not limited to, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

In some embodiments, the present disclosure provides a method for reducing scarring of wounds, the method comprising: applying a biophotonic composition of the present disclosure to a site of the wound (e.g., topical application of the biophotonic composition so as to cover at least the entirety of the wound), and illuminating the applied biophotonic composition with light having a wavelength that overlaps with an absorption spectrum of the at least one light-accepting molecule of the biophotonic composition.

In some instances, the method of the present disclosure is performed at least once weekly. That is to say that every week, the biophotonic composition is applied onto the wound and the applied biophotonic composition is illuminated for a period of at least 5 minutes.

In other instances, the method of the present disclosure is performed at least twice weekly. In some of these instances, once the method as been performed once, the used biophotonic composition is removed from the wound. Then, right after removal of the used biophotonic composition, the method is performed again, and a further/fresh amount of biophotonic composition is applied onto the wound and the fresh amount of biophotonic composition is illuminated for a period of at least 5 minutes. In some other instances, however, the further/fresh amount of biophotonic composition is only applied after a rest period. For example, wherein the first occurrence of the method is performed on day 1, the second occurrence of the method may be performed on day 2, or on day 3, or on day 4, or on day 5, or on day 6 or on day 7. In some instances, the first occurrence of the method of the present disclosure may be performed (initiated) as early as 1 day, 2 days, 3 days, 4 days, 5, days, 6 days, 7 days, 8 days, 9 days, 10 days, 11 days, 12 days, 13 days, 14 days, 15 days, 16 days, 17 days, 18 days, 20 days or 21 days following closure of the wound.

In some instances, the methods of the present disclosure may be performed (initiated) as soon as the wound is closed.

In some other instances, the methods of the present disclosure may be performed (initiated) as soon as scarring is initiated.

In some instances, the first occurrence of the method of the present disclosure is performed (initiated) as early as 7 days following occurrence of the wound (e.g., surgery).

In some instances, the first occurrence of the method of the present disclosure is performed (initiated) as early as 21 days following occurrence of the wound (e.g., surgery).

Alternatively, the method of the present disclosure may be initiated when suitable closure of the wound is achieved.

In some embodiments, the method of the present disclosure is performed over a period of at least about 4 weeks. In some instances, the method of the present disclosure is performed over a period that spans between about 4 weeks to about 24 weeks. Alternatively, the method of the present disclosure may be performed until a satisfactory level of reduction of scarring is achieved such as by suturing of the wound.

In the methods of the present disclosure, any source of actinic light can be used. Any type of halogen, LED or plasma arc lamp or laser may be suitable. In some instances, the light is a continuous light. In some other instances the light is modulated. The primary characteristic of suitable sources of actinic light will be that they emit light in a wavelength (or wavelengths) appropriate for activating the one or more photoactivators present in the composition. In one embodiment, an argon laser is used. In another embodiment, a potassium-titanyl phosphate (KTP) laser (e.g. a GreenLight™ laser) is used. In another embodiment, sunlight may be used. In yet another embodiment, a LED photocuring device is the source of the actinic light. In yet another embodiment, the source of the actinic light is a source of light having a wavelength between about 200 to 800 nm. In another embodiment, the source of the actinic light is a source of visible light having a wavelength between about 400 and 600 nm. Furthermore, the source of actinic light should have a suitable power density. Suitable power density for non-collimated light sources (LED, halogen or plasma lamps) are in the range from about 1 mW/cm² to about 200 mW/cm². Suitable power densities for laser light sources are in the range from about 0.5 mW/cm² to about 0.8 mW/cm².

In some embodiments of the methods of the present disclosure, the light has an energy at the subject's skin/soft tissue of between about 1 mW/cm² and about 500 mW/cm², 1-300 mW/cm², or 1-200 mW/cm², wherein the energy applied depends at least on the wavelength of the light, the distance of the subject's skin/soft tissue from the light source, and the thickness of the biophotonic composition. In certain embodiments, the light at the subject's skin/soft tissue is between about 1-40 mW/cm², or 20-60 mW/cm², or 40-80 mW/cm², or 60-100 mW/cm², or 80-120 mW/cm², or 100-140 mW/cm², or 120-160 mW/cm², or 140-180 mW/cm², or 160-200 mW/cm², or 110-240 mW/cm², or 110-150 mW/cm², or 190-240 mW/cm².

In certain embodiments, different sources of light can be used to activate the biophotonic compositions, such as a combination of ambient light and direct LED light.

The duration of the illumination period (during which the biophotonic composition is exposed to light) required may depend on the surface of the treated area, the type of wound that is being treated, the power density, wavelength and bandwidth of the light source, the thickness of the biophotonic composition, and the treatment distance from the light source.

The illumination of the wound may take place within seconds or even fragment of seconds, but a prolonged illumination period is beneficial to exploit the synergistic effects of the absorbed, reflected and reemitted light on the biophotonic composition of the present disclosure and its interaction with the skin/soft tissue being treated.

In one embodiment, the illumination period of the biophotonic composition applied to the wound is at least about 5 minutes. In another embodiment, the illumination period of the biophotonic composition applied to the wound is between about 5 minutes and 30 minutes. In certain embodiments, light is applied for a period of 5-10 minutes, 10-15 minutes, 15-20 minutes, 20-25 minutes, or 20-30 minutes. In some embodiments, a fresh application of the biophotonic composition is applied before exposure to actinic light.

In the methods of the present disclosure, the biophotonic composition may be optionally removed from the wound following application of light. In certain embodiments, the biophotonic composition is left on the wound for more than 30 minutes, more than one hour, more than 2 hours, more than 3 hours. It can be illuminated with ambient light. To prevent drying, the composition can be covered with a transparent or translucent cover such as a polymer film, or an opaque cover which can be removed before illumination.

Acute wounds are typically categorized based on causes (lacerations, abrasions, punctures, incisions, gunshots and burns), and type according to size and depth (superficial or deep). In some instances, acute wounds include post-surgical wounds and traumatic wounds. Acute wounds include abrasion wounds, laceration wounds, puncture wounds, incision wounds, and gunshot wounds.

Abrasion wounds are caused by friction with other surfaces or objects that scrape off the top layers of skin (as can occur after falling down while running). Abrasions are shallow and usually irregular in shape, with some pain and little to no bleeding.

Laceration wounds are tear-like wounds deeper than abrasions, resulting from a blunt trauma or blow from objects, collisions or accidents. The skin is usually torn irregularly and there is more pain and bleeding than seen in abrasions.

Puncture wounds are small rounded wounds that result from penetrating objects such as needles or nails. The wound is typically the same size and shape as the causative object. Bleeding and pain are minor and subside shortly after removing the object.

Incision wounds are clean cuts that result from sharp objects like knives, scissors and scalpels. Incisions are linear with regular edges, and can be superficial (limited to the uppermost skin layers) or deep (reaching the muscles and underlying organs). Incision wounds are very painful and can be life-threatening, especially if they involve a vital organ (like the heart or lungs) or major blood vessels.

Gunshot wounds are caused by firearms; with regular, rounded edges smaller than the bullet at point of entrance. The wound may have burn marks or soot on the edges and surrounding tissue, depending on the distance of the gun from the skin when it was fired. If the bullet goes all the way through the body, the exit wound will be irregular in shape and larger than the entrance wound, with more bleeding. Bullets move in a straight line except when they hit a bone; in which case they can break through and shatter the bone, or be deflected in another direction. Apart from the risk of hitting vital organs or major blood vessels, the fast spiraling movement of the bullet can cause serious damage to the tissue it passes through.

Once cleaned, acute wounds are preferably closed with, for examples, stitches, staples, skin adhesive bands, and dressing with a sterile bandage, with or without application of topical antibiotic ointment or with skin grafting (covering the wound area with healthy skin taken from other parts of the body). Wound closure is necessary to bring the separated tissue together and cover the exposed tissue to reduce the risk of infection and promote healing.

The patient and observer scar assesment scale (POSAS) assess the most commonly described scar characteristics from a patient and observers perspective. Scar characteristics include, but are not limited to, vascularity, pigmentation, relief/texture, thickness, pliability, surface area, pain and itching/pruritus. The increased vascularisation of the scar (erythema) is a good indicator for scar activity in the early maturation phase. On the long term scars frequently become pale. Pigmentation disorders are caused by variation in the concentration of melanocytes in the epidermal layer and their melanin production. Significant pigmentation disorders may remain in the long term. Relief/texture relates to irregularities of the scar surface (surface roughness or relief) are particularly seen after split skin autografting by using a meshed split skin graft. The irregularities result from secondary healing of the interstices of the meshed skin graft. Scar tissue normally becomes thicker (thickness) than the surrounding skin (hypertrophy) during the first months after which the thickness reduces in most cases. Burn scars frequently remain hypertrophic to some extent but scar atrophy is also noted in some scar categories. In daily clinical practice, normally the protruding part of the scar, compared to the surrounding skin, is judged. Scar tissue is normally less supple (pliability) than normal skin mainly because the scar is thicker and has an inferior quality of collagen architecture. This may cause functional impairment, especially when scars are located on or around joints. The surface area of scars can either reduce or increase as a result of scar contraction or expansion respectively. Scar contraction is mostly considered as a problem in burn scars where it may cause functional problems, whereas scar expansion or widening is often observed in linear scarring. The pathophysiology is still not well understood, but a strong relation with scar hypertrophy and itching has been reported. Itching/pruritus is an irritating cutaneous sensation that produces a desire to scratch. The impact of itching caused by scar tissue is frequently underestimated especially when large body surface areas are involved i.e. after extensive burn injuries or paediatric burn injury. Itching is often associated with hypertrophic scarring.

In some embodiments, the method of the present disclosure reduces scarring of wounds.

In some instances, the method of the present disclosure increases vascularity of the wound. In some other instances, the method of the present disclosure restores pigmentation of the skin/soft tissue of the wound towards pigmentation of the normal (non-wounded) skin around the wound.

In some other instances, the method of the present disclosure decreases thickness of the wound.

In some other instances, the method of the present disclosure increases pliability of the skin/soft tissue comprising the wound.

In some other instances, the method of the present disclosure reduces the surface area of the wound.

In some other instances, the method of the present disclosure reduces itching and/or pruritus associated with the wound.

Several methodologies and modalities have been devised to quantify scars for the purposes of determining response to treatment and for evaluating outcomes. Scar assessments can be objective or subjective. Objective assessments provide a quantitative measurement of the scar, whereas subjective assessments are observer dependent. Quantitative assessment of scars requires devices to measure their physical attributes. Subjective methods to assess scar provide a qualitative measurement of scar by a patient or clinician. Scar-measuring devices should be noninvasive, accurate, reproducible, and easy-to-use to facilitate objective data collection and have clinical utility. Existing devices assess parameters such as pliability, firmness, color, perfusion, thickness, and 3-dimensional topography.

Several tools have been applied to assess pliability including the pneumatonometer and cutometer. The pneumatonometer uses pressure to objectively measure skin pliability. It is composed of a sensor, a membrane, and an air-flow system that measures the amount of pressure needed to lock the system. Application of the pneumatonometer to measure cutaneous compliance (Δ volume/Δ pressure) has yielded statistically significant differences in skin compliance based on body site as well as demonstrated overall less compliance of burn scars in all sites as compared to normal controls. The cutometer is a noninvasive suction device that has been applied to the objective and quantitative measurement of skin elasticity. It measures the viscoelasticity of the skin by analyzing its vertical deformation in response to negative pressure. It has been used to measure the effects of treatments on burn scars and to assess scar maturation. The durometer applies a vertically directed indentation load on the scar to measure tissue firmness. Tools have also been developed to objectively measure scar color. The Chromameter (Minolta, Tokyo, Japan), the DermaSpectrometer (cyberDERM, Inc, Media, PA, USA), the Mexameter (Courage-Khazaka, Cologne, Germany), and the tristimulus colorimeter are among the most widely applicable devices. These devices use spectrophotometric color analysis to calculate erythema and melanin index. Ultrasound scanners, such as the tissue ultrasound palpation system (TUPS), have been used to quantify scar thickness. Laser Doppler perfusion imaging is an established technique for the measurement of burn scar perfusion. It aids in early determination of burn depth and subsequent treatment course. Through constructing color-coded maps of tissue microperfusion, laser Doppler perfusion imaging offers a noninvasive alternative to burn wound biopsy. Three-dimensional systems are used for their ability to capture scar surface characteristics with high definition and reproducibility.

Scar scales have been devised to quantify scar appearance in response to treatment. There are currently at least 5 scar scales that were originally designed to assess subjective parameters in an objective way: The Vancouver Scar Scale (VSS), Manchester Scar Scale (MSS), Patient and Observer Scar Assessment Scale (POSAS), Visual Analog Scale (VAS), and Stony Brook Scar Evaluation Scale (SBSES). These observer-dependent scales consider factors such as scar height or thickness, pliability, surface area, texture, pigmentation, and vascularity. Table 1 provides a comparison of scar assessment scales.

TABLE 1
Comparison of scar assessment scale

| | Scoring | |
|---|---|---|
| Scale | System | Attributes analyzed |
| Vancouver | 0 to 13 | Vascularity, height/thickness, |
| Scar Scale | | pliability, and pigmentation |
| (VSS) | | |
| Visual Analog | 0 to 100 | Vascularity, pigmentation, |
| Scale with | “excellent” | acceptability, observer comfort plus |
| Scar Ranking | to “poor” | contour and summing the individual |
| (VAS) | | scores |
| Patient and | 6 to 60 | VSS plus surface area; patient |
| Observer Scar | | assessments of pain, itching, color, |
| Assessment | | stiffness, thickness, relief |
| Scale | | |
| Manchester | 5 (best) | VAS plus scar color, skin texture, |
| Scar Scale | to 18 (worse) | relationship to surrounding skin, |
| | | texture, margins, size, multiplicity |
| Stony Brook | 0 (worse) | VAS plus width, height, color, |
| Scar | to 5 (best) | presence of suture/staple marks |
| Evaluation | | |
| Scale | | |

The VSS assesses 4 variables: vascularity, height/thickness, pliability, and pigmentation. Patient perception of his or her respective scars is not factored in to the overall score. The VSS ranges from 0 to 13 where 0 is the least amount of scarring and 13 is the most amount of scarring. Table 2 indicates the correspondence between the scoring on VSS and evolution of the variables assessed by the test.

TABLE 2
Scoring on VSS

| | |
|---|---|
| Vascularity | 0 pt = normal color that closely resembles the color |
| | over the rest of the body. |
| | 1 pt = pink. This color, usually observed during a |
| | transient period of time in most of the normal |
| | maturation processes, becomes a warning signal of |
| | pathological scar when it remains present after the |
| | second month of evolution. |
| | 2 pt = red. A red aspect is linked to a scar hyper- |
| | vascularization. This color becomes obvious 4 to |
| | 8 weeks after complete healing and is a good sign |
| | of pathologic evolution. Usually, a red scar is |
| | combined with a progressive elevation, defining a |
| | scar hypertrophy. |
| | 3 pt = purple. A purple scar is observed in highly |
| | vascularized scars, like burn scars or at the initial |
| | stage of a keloid process. |
| Pigmentation | 0 pt = normal skin |
| | 1 pt = hypopigmentation |
| | 2 pt = hyperpigmentation |
| Pliability | 0 pt = normal, |
| | 1 pt = supple: flexible with minimal resistance, |
| | 2 pt = yielding: giving way to pressure, |
| | 3 pt = firm: inflexible, not easily moved, resistant |
| | to manual pressure, |
| | 4 pt = banding: producing striations which blanch |
| | on stretching but with no limit to the range of |
| | motion, |
| | 5 pt = contracture of any type of scar limiting the |
| | range of motion. |
| Height | 0 pt = normal |
| | 1 pt = height (h) < 2 mm |
| | 2 pt = 2 mm < h < 5 mm |
| | 3 pt = h > 5 mm |

Total possible score = 13

The POSAS includes subjective symptoms of pain and pruritus and expands on the objective data captured in the VSS. It consists of 2 numerical numeric scales: The Patient Scar Assessment Scale and the Observer Scar Assessment Scale. It assesses vascularity, pigmentation, thickness, relief, pliability, and surface area, and it incorporates patient assessments of pain, itching, color, stiffness, thickness, and relief. The POSAS is the only scale that considers subjective symptoms of pain and pruritus, but like other scales it also lacks functional measurements as to whether the pain or pruritus interferes with quality of life. Linear regression analysis has demonstrated that the observer's opinion is influenced by vascularization, thickness, pigmentation, and relief, whereas the patient's opinion is primarily influenced by pruritus and scar thickness. The POSAS has been applied to postsurgical scars and used in the evaluation of linear scars following breast cancer surgery, demonstrating internal consistency and interobserver reliability when compared to the VSS with the added benefit of capturing the patients' ratings. Table 3 indicates the correspondence between the scoring on the Observer POSAS and evolution of the variables assessed by the test, whereas Table 4 indicates the correspondence between the scoring on the Patient POSAS and evolution of the variables assessed by the test.

TABLE 3
Scoring on Observer POSAS
Each item has a 10-point score, with 10 indicating the
worst cars or sensation. The lowest score is 1 and
corresponds to a situation of normal skin property.

| | |
|---|---|
| Vascularity | 1 to 10 = Presence of vessels in scar tissue assessed by |
| | the amount of redness, tested by the amount of blood |
| | return after blanching with a piece of Plexiglas |
| Pigmentaion | 1 to 10 = Brownish coloration of the scar by pigment |
| | (melanin); apply Plexiglas to he skin with moderate |
| | pressure to eliminate the effect of vascularity |
| Thickness | 1 to 10 = Average distance between the subcuticular- |
| | dermal border and the epidermal surface of the scar |
| Relief | 1 to 10 = The extent to which surface irregularities |
| | present (preferably compared with adjacent normal |
| | skin) |
| Pliability | 1 to 10 = Suppleness of the scar tested by wrinkling |
| | the scar between the thumb and index finger |
| Surface Area | 1 to 10 = Surface area of the scar in relation to the |
| | original wound area |

Total score = between 6 and 60

TABLE 4
Scoring on Patient POSAS
Each question has a 10-point score, with 10 indicating
the worst cars or sensation. The lowest score is 1 and
corresponds to a situation of normal skin property.

| | |
|---|---|
| Pain | 1 to 10 = Has the scar been painful the last few weeks? |
| Itching | 1 to 10 = Has the scar been itching the last few weeks? |
| Color | 1 to 10 = Is the scar color different from the color of the |
| | normal skin at the present? |
| Pliability | 1 to 10 = Is the stiffness of the scar different from your |
| | normal skin at the present? |
| Thickness | 1 to 10 = Is the thickness of the scar different from your |
| | normal skin at the present? |
| Relief | 1 to 10 = IS the scar more irregular than your normal |
| | skin at the present? |

Total score = between 6 and 60

The present disclosure also provides kits for reducing scarring of a wound. In particular, the kit is for preparing and/or applying any of the biophotonic compositions of the present disclosure to an wound. The kit may include a biophotonic topical biophotonic composition, as defined above, together with one or more of a light source, devices for applying or removing the biophotonic composition, instructions of use for the biophotonic composition and/or light source.

In some embodiments, the biophotonic composition comprises at least a first light-accepting molecule in a gelling agent. The light-accepting molecule may be present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the biophotonic composition. In embodiments where the biophotonic composition comprises more than one light-accepting molecule, the first light-accepting molecule may be present in an amount of between about 0.01-40% per weight of the composition, and a second light-accepting molecule may be present in an amount of between about 0.0001-40% per weight of the composition.

In certain embodiments, the first light-accepting molecule is present in an amount of between about 0.01-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the composition. In certain embodiments, the second light-accepting molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the composition. In certain embodiments, the amount of light-accepting molecule or combination of light-accepting molecules may be in the amount of between about 0.05-40.05% per weight of the composition. In certain embodiments, the amount of light-accepting molecule or combination of light-accepting molecules may be in the amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40.05% per weight of the composition. The composition may include an oxygen-releasing agent present in amount between about 0.01%-40%, between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, or between about 30%-40% by weight to weight of the composition. Alternatively, the kit may include the oxygen-releasing agent as a separate component to the light-accepting molecule containing composition.

In some embodiments, the kit includes more than one composition, for example, a first and a second composition. The first composition may include the oxygen-releasing agent and the second composition may include the first light-accepting molecule in the gelling agent. The first light-accepting molecule may have an emission wavelength between about 400 nm and about 570 nm. The oxygen-releasing agent may be present in the first composition in an amount of between about 0.01%-1.0%, between about 0.5%-10.0%, between about 5%-15%, between about 10%-20%, between about 15%-25%, between about 20%-30%, between about 15.0%-25%, between about 20%-30%, between about 25%-35%, between about 30%-40% or between about 35%-45% by weight to weight of the first composition. The light-accepting molecule may be present in the second composition in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the second composition. In embodiments where the second composition comprises more than one light-accepting molecule, the first light-accepting molecule may be present in an amount of between about 0.01-40% per weight of the second composition, and a second light-accepting molecule may be present in an amount of about 0.0001-40% per weight of the second composition. In certain embodiments, the first light-accepting molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the second composition. In certain embodiments, the second light-accepting molecule is present in an amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the second composition. In certain embodiments, the amount of light-accepting molecule or combination of light-accepting molecules may be in the amount of about 0.05-40.05% per weight of the second composition. In certain embodiments, the amount of light-accepting molecule or combination of light-accepting molecules may be in the amount of between about 0.001-0.1%, between about 0.05-1%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40.05% per weight of the second light-accepting molecule.

In some other embodiments, the first composition may comprise the first light-accepting molecule in a liquid or as a powder, and the second composition may comprise a gelling composition for thickening the first composition. The oxygen-releasing agent may be contained in the second composition or in a third composition in the kit. In some embodiments, the kit includes containers comprising the compositions of the present disclosure. In some embodiments, the kit includes a first container comprising a first composition that includes the oxygen-releasing agent, and a second container comprising a second composition that includes at least one light-accepting molecule. The containers may be light impermeable, air-tight and/or leak resistant. Exemplary containers include, but are not limited to, syringes, vials, or pouches. The first and second compositions may be included within the same container but separated from one another until a user mixes the compositions. For example, the container may be a dual-chamber syringe where the contents of the chambers mix on expulsion of the compositions from the chambers. In another example, the pouch may include two chambers separated by a frangible membrane. In another example, one component may be contained in a syringe and injectable into a container comprising the second component.

Written instructions on how to use the biophotonic composition in accordance with the present disclosure may be included in the kit, or may be included on or associated with the containers comprising the compositions of the present disclosure.

In certain embodiments, the kit may comprise a further component which is a dressing. The dressing may be a porous or semi-porous structure for receiving the biophotonic composition. The dressing may comprise woven or non-woven fibrous materials.

In certain embodiments of the kit, the kit may further comprise a light source such as a portable light with a wavelength appropriate to activate the light-accepting molecule in the biophotonic composition. The portable light may be battery operated or re-chargeable.

In certain embodiments, the kit may further comprise one or more waveguides.

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLE

The example below is given so as to illustrate the practice of various embodiments of the present disclosure. It is not intended to limit or define the entire scope of this disclosure.

### Evaluation of a Biophotonic Treatment and Comparison with Silicone Sheet in the Reduction of Scarring of Surgical Wounds in Human Subjects

Overall Study Design:

The study was performed with 42 patients who had just been through bilateral breast reduction. The evaluation of the efficacy of the biophotonic treatment compared to the treatment using silicone sheets in reducing scarring of the surgical wounds was assessed using the following assessment scales:
Physician Observer Scar Assessment Scale (POSAS) as well as Patient Observer Scar Assessment Scale (POSAS) at Weeks 1, 4, 8, 12, 18 and 24, post-surgery;
Vancouver Scar Assessment Scale (VSAS);
Patient's self-assessment of ease of wound management; and
Blinded experts panel assessment of wound appearance (following completion).

Comparison A:

Patients having gone through breast reduction and randomized to Comparison A were re-randomized to get a biophotonic treatment using the biophotonic composition according to one embodiment of the present disclosure (wherein the biophotonic composition comprised: Eosin Y at a concentration of 0.305 mg/ml and carbamide peroxide at a concentration of 12%) which was applied once weekly (Group A1) or twice weekly (Group A2) starting on Day 7 post-surgery (breast reduction). The breast wound receiving the biophotonic treatment was randomly selected. The breast wound receiving silicone sheets was also randomly selected and received a first application of silicone sheets on Day 21 post-surgery. The biophotonic treatment was applied for a minimum period of 6 weeks, up to a maximum period of 8 weeks. The silicone sheets were applied for a minimum period of 8 weeks and up to a maximum period of 12 weeks.

Comparison B:

Patients having gone through breast reduction and randomized to Comparison B were re-randomized to get a biophotonic treatment using the biophotonic composition according to one embodiment of the present disclosure (wherein the biophotonic composition comprised: Eosin Y at a concentration of 0.305 mg/ml and carbamide peroxide at a concentration of 12%) which was applied once weekly (Group B1) or twice weekly (Group B2) starting on Day 21 post-surgery (breast reduction). The breast wound receiving the biophotonic treatment was randomly selected. The breast wound receiving silicone sheets was also randomly selected and received a first application of silicone sheets on Day 21 post-surgery. The biophotonic treatment was applied for a minimum period of 6 weeks, up to a maximum period of 8 weeks. The silicone sheets were applied for a minimum period of 8 weeks and up to a maximum period of 12 weeks.

Comparison C:

Patients having gone through breast reduction and randomized to Comparison C were re-randomized to get double (two consecutive treatments) the biophotonic treatment using the biphotonic composition according to one embodiment of the present disclosure (wherein the biophotonic composition comprised: Eosin Y at a concentration of 0.305 mg/ml and carbamide peroxide at a concentration of 12%) which was applied once weekly starting either at Day 7 (Group C1) or at Day 21 (Group C2) post-surgery (breast reduction). The breast wound receiving the biophotonic treatment was randomly selected. The breast wound receiving silicone sheets was also randomly selected and received a first application of silicone sheets on Day 21 post-surgery (breast reduction). The double biophotonic treatment was applied for a minimum period of 6 weeks, up to a maximum period of 8 weeks. The silicone sheets were applied for a minimum period of 8 weeks and up to a maximum period of 12 weeks.

Biophotonic Treatment:

An amount of the biophotonic composition was topically applied onto the area of the breast having the acute wound and the biophotonic composition was illuminated for a period of 5 minutes using a phototherapeutic lamp. Following the 5 minute illumination period, the biophotonic composition was removed (e.g., washed off) from the skin. For patients receiving the double biophotonic treatment (two consecutive biophotonic treatments (Comparison C)), once the first illumination of the acute wound was performed, the used biophotonic composition was removed and a second amount of fresh biophotonic composition was applied right away onto the same treatment area. The second application of fresh biophotonic composition was then illuminated for 5 minutes using the phototherapeutic lamp. The silicone sheets were used and applied as per manufacturers' instructions.

Phototherapeutic Lamp:

The study was performed using a phototherapeutic device delivering non-coherent blue light with peak wavelengths in the range of 440-460 nm, an irradiance or power density of between 55 and 129 mW/cm² at a distance of 5 cm (distance between the light source and the surface to the illuminated) source with a radiant fluence (or dose) during a single treatment for 5 minutes of 16.5 to 38.7 J/cm².

Silicone Sheets (CICA-CARE® SILICONE SHEETING):

CICA-CARE® SILICONE SHEETING is a self-adhesive silicone gel sheet medically proven to be up to 90% effective in the improvement of red, dark or raised scars. It is designed for use in the management of both existing and new hypertrophic and keloid scars (red and raised) and as a preventive therapy on closed wounds to prevent hypertrophic and keloid scars (red and raised).

Biophotonic Treatment Period:

Patients could be randomized to one of three different comparisons:
Comparison A: Application of the biophotonic composition initiated on Day 7 post-surgery, once or twice weekly for a period of 6-8 weeks, vs. silicone sheets initiated on Day 21 post-surgery for a period of 8-12 weeks;
Comparison B: Application of the biophotonic composition initiated on Day 21 post-surgery, once or twice weekly for a period of 6-8 weeks, vs. silicone sheets initiated on Day 21 post-surgery for a period of 8-12 weeks; and
Comparison C: Double Application of the biophotonic composition initiated on Day 7 or on Day 21 post-surgery, once weekly for a period of 6-8 weeks, vs. silicone sheets initiated on Day 21 post-surgery for a period of 8-12 weeks.

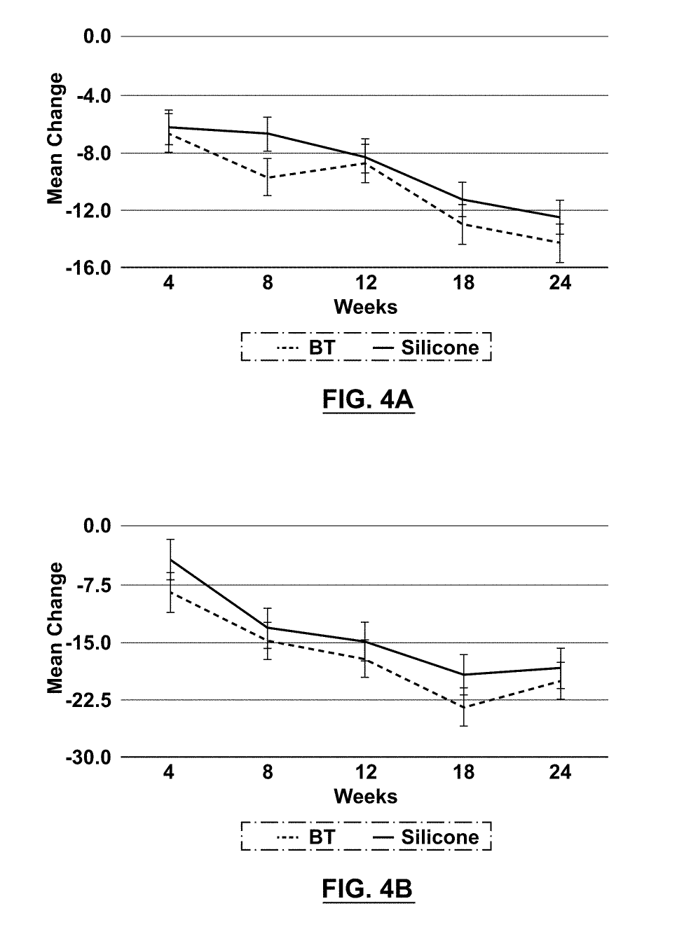

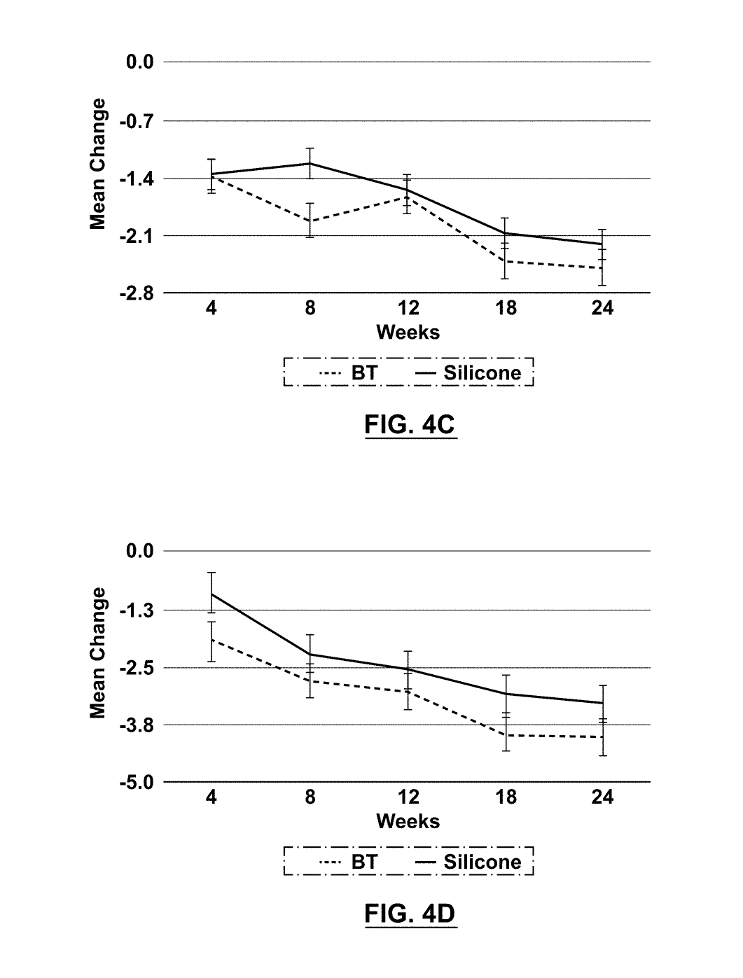

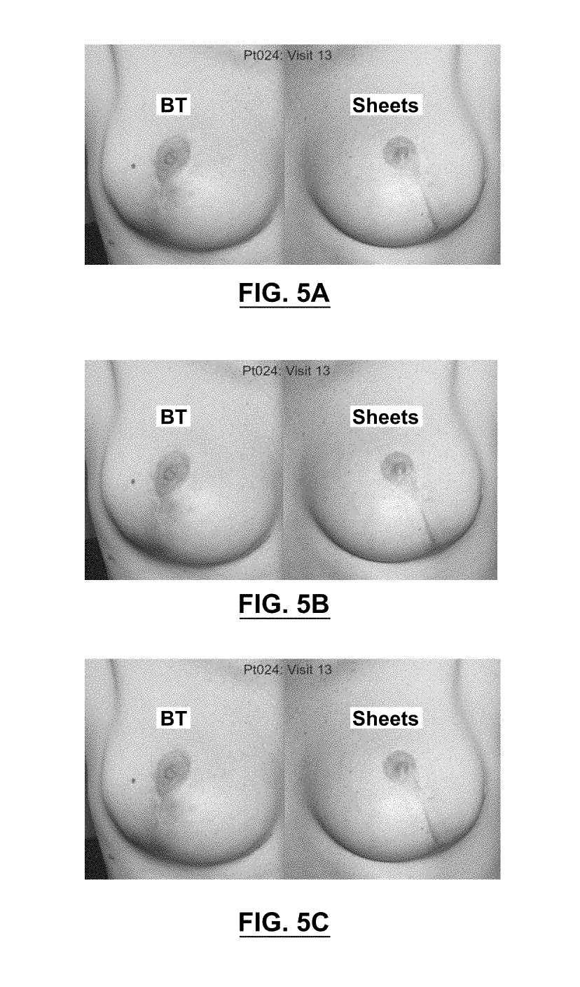

Overall, as shown by the Total Score (Observers: FIG. 4A; Patient: FIG. 4B) and the Overall opinion (Observers: FIG. 4C; Patient: FIG. 4D) obtained for all of Groups (A1+A2+B1+B2+C1+C2) demonstrate that the biophotonic treatment was as good as or better than treatment with the silicone sheets.

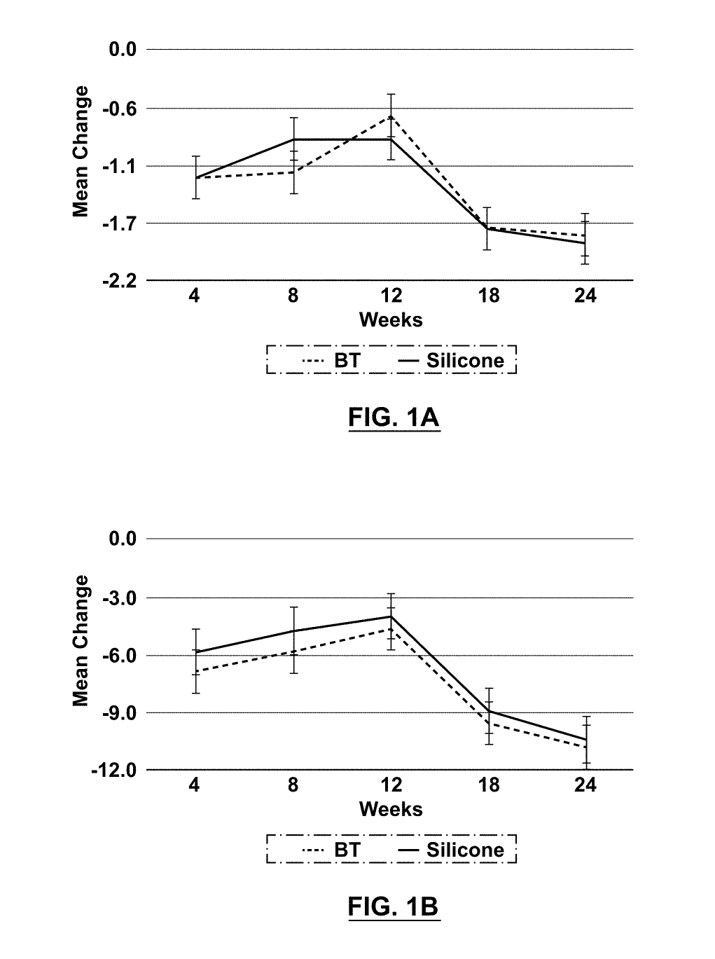

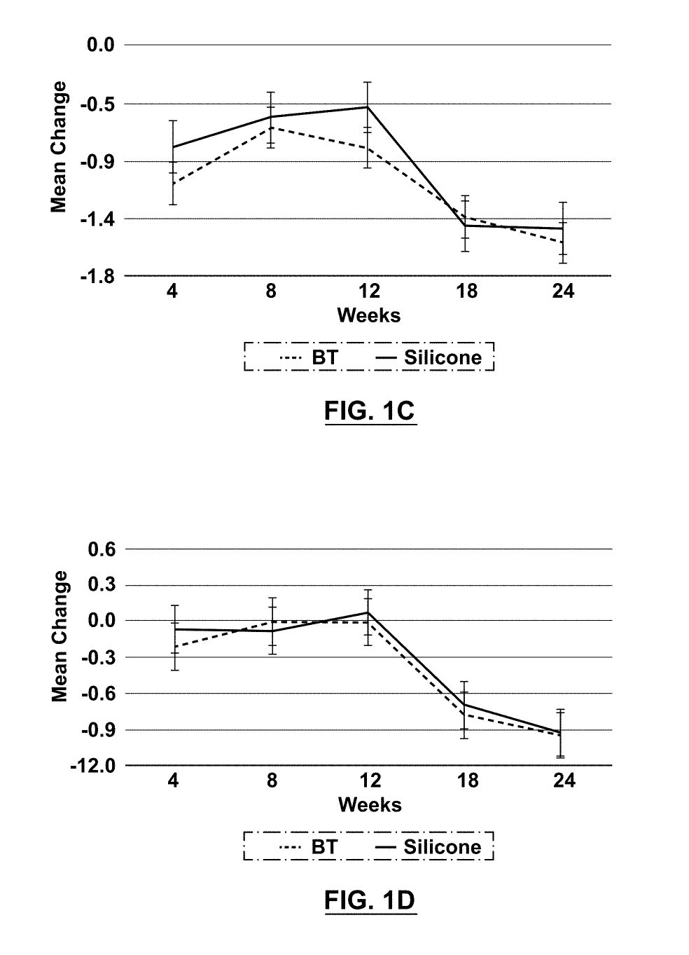

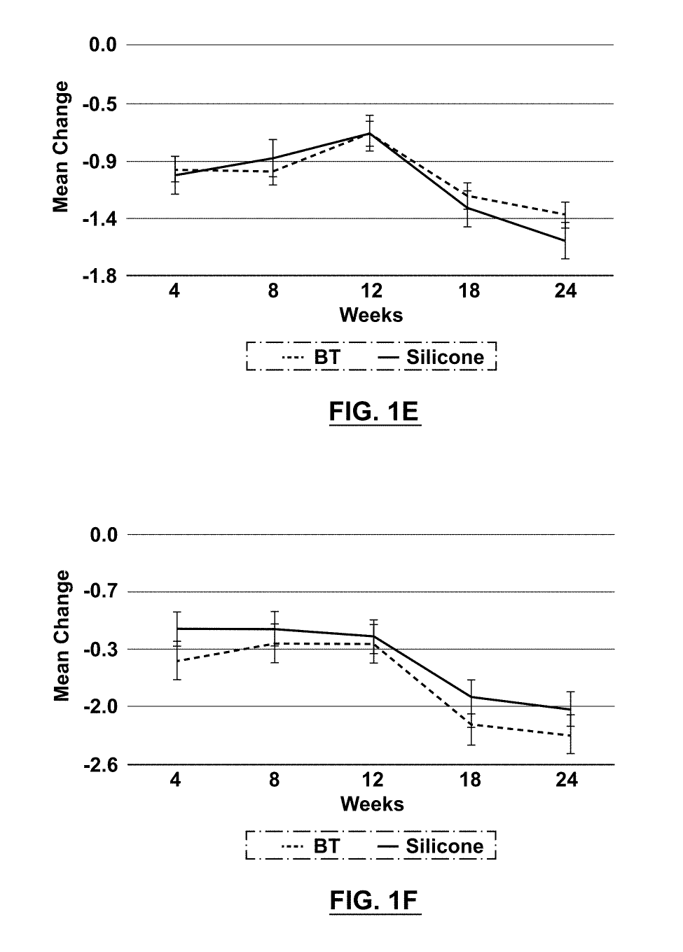

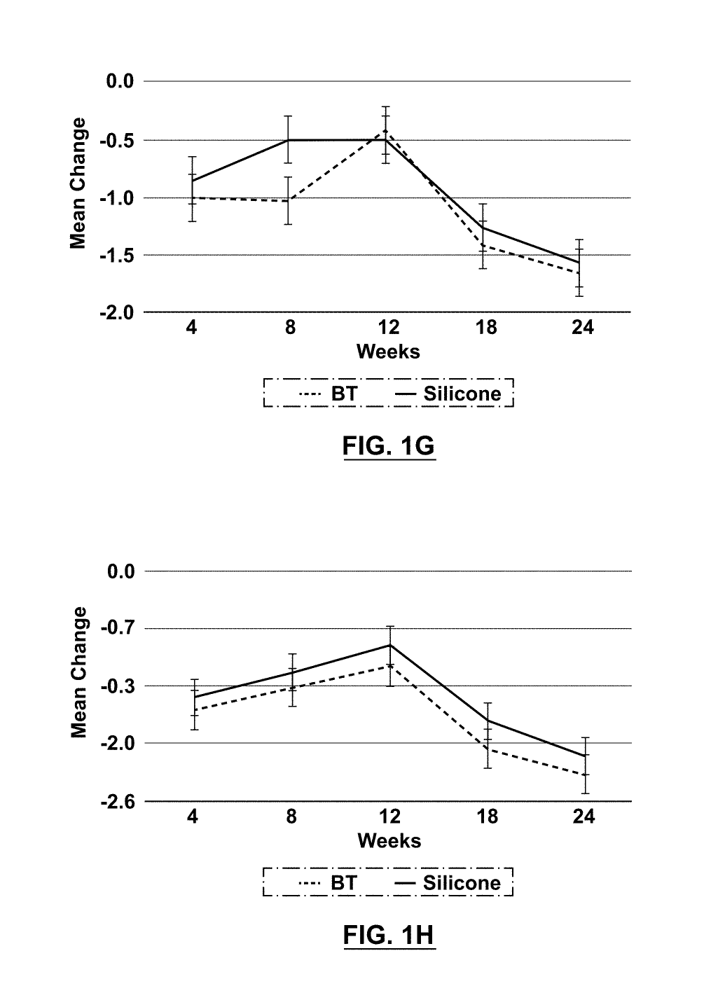

In particular, patients in Group A1 and Group B1 showed reduced scarring of the acute wound at week 4 post-surgery compared to week 0 (baseline) (FIG. 1A and FIG. 1B), indicating that a single biophotonic treatment once a week for a period of about 4 weeks is efficient to reduce scarring. At week 4 post-surgery, for patients in Group A1 and Group B1 showed an improved restoration of healthy skin properties such as, vascularity (FIG. 1C), pigmentation (FIG. 1D), skin thickness (FIG. 1E), pliability (FIG. 1EF) and surface area (FIG. 1G) compared to baseline. Relief was also observed in patients treated with the biophotonic treatment (FIG. 1H). Restoration of skin properties using the biophotonic treatment was as good as or better than treatment with silicone sheets.

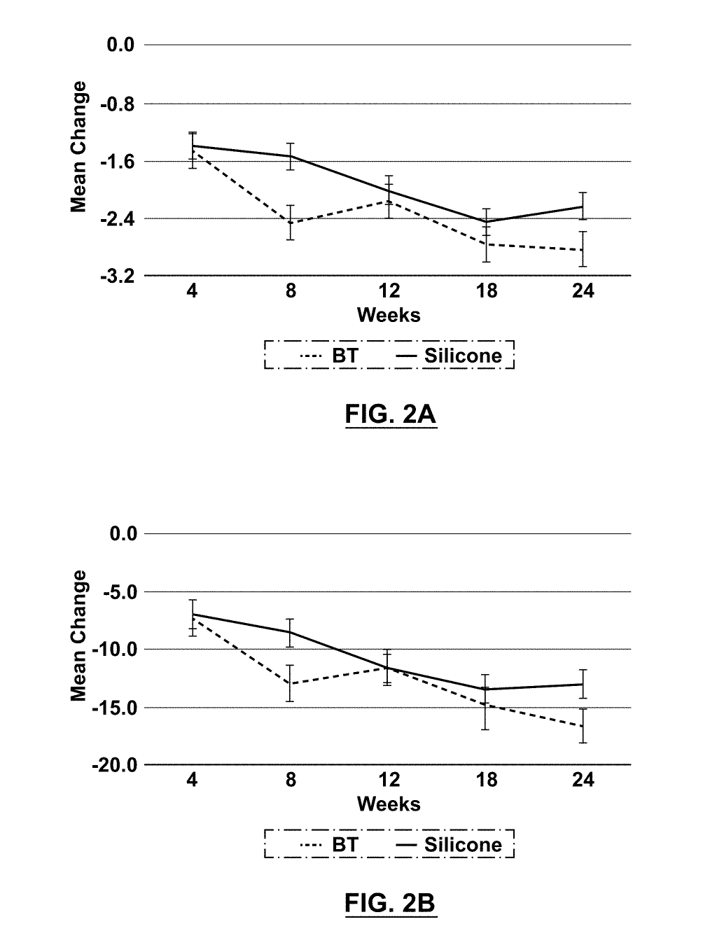

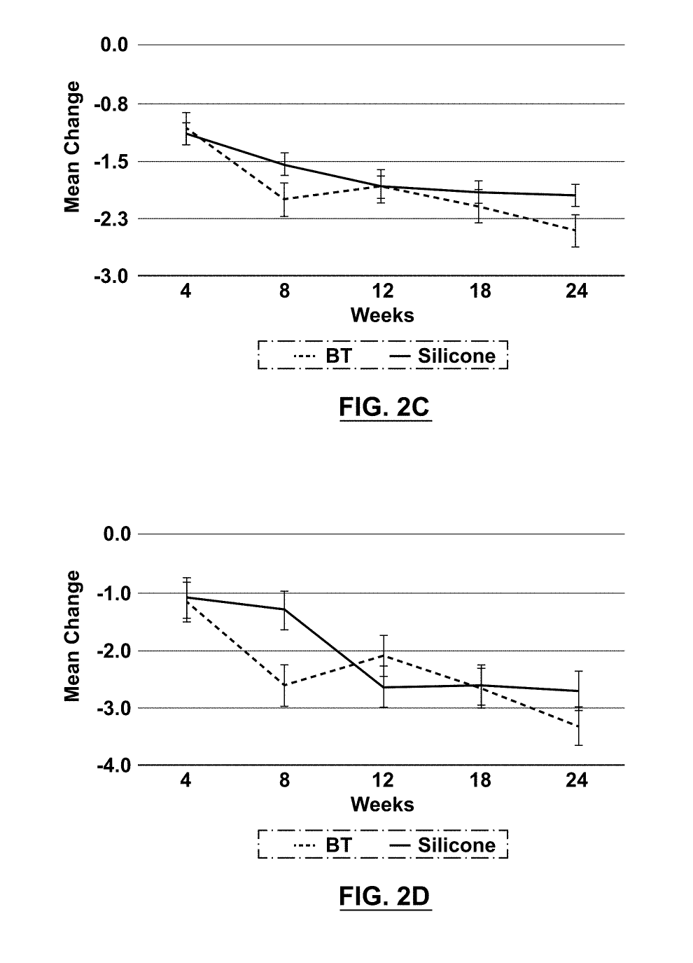

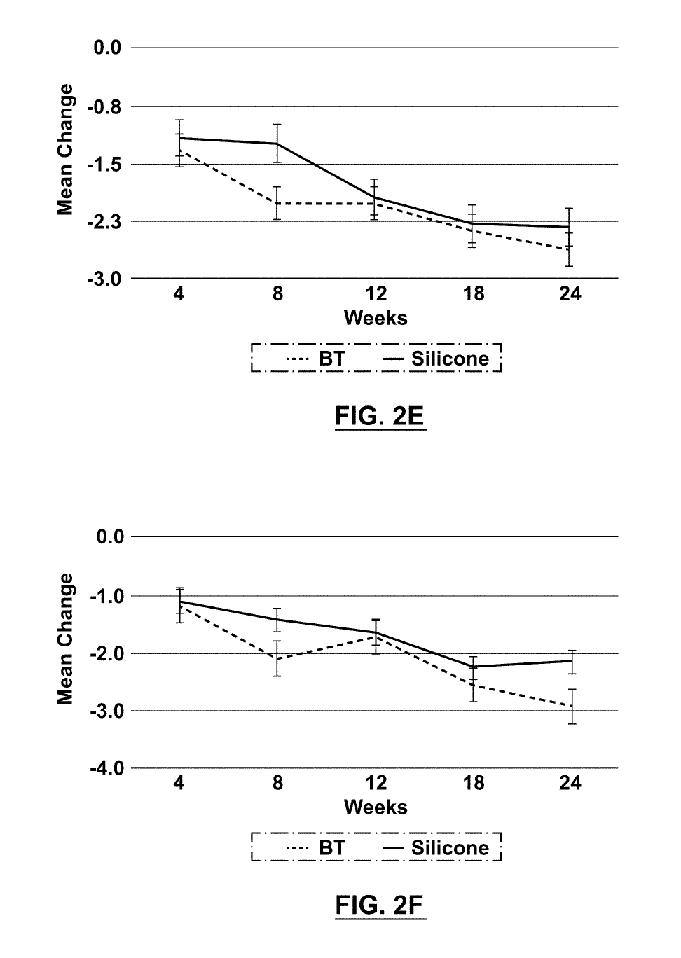

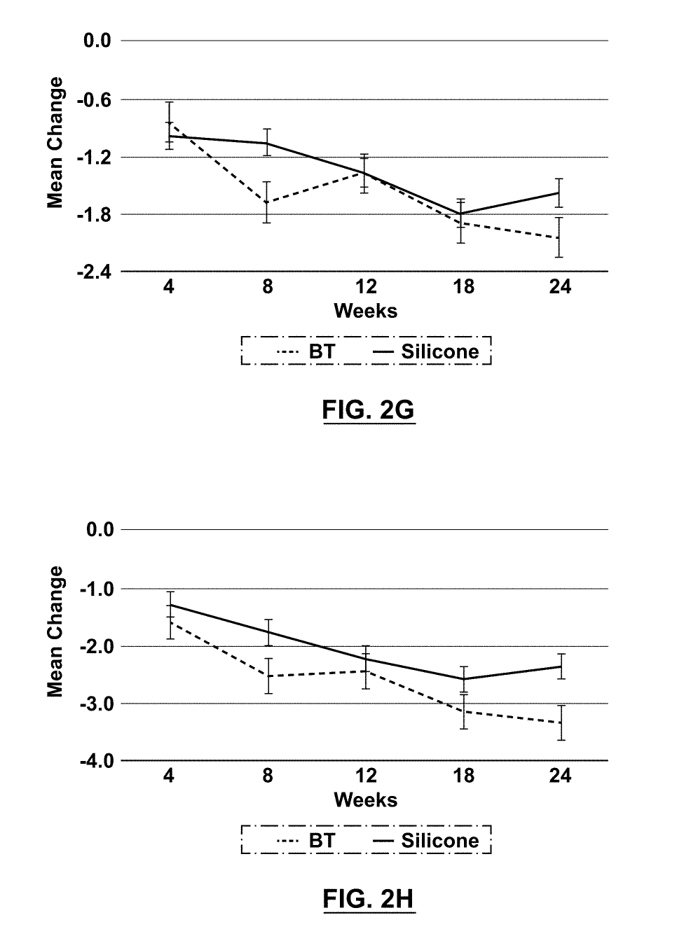

Patients in Group A2 and Group B2 showed reduced scarring of the acute wound at week 4 post-surgery compared to week 0 (baseline) (FIG. 2A and FIG. 2B), indicating that a single biophotonic treatment twice a week for a period of about 4 weeks is efficient to reduce scarring. At 4 weeks, patients in Group A2 and Group B2 observed improved restoration of healthy skin properties such as, vascularity (FIG. 2C), pigmentation (FIG. 2D), skin thickness (FIG. 2E), pliability (FIG. 2F) and surface area (FIG. 2G) compared to baseline. Relief was also observed in patients treated with the biophotonic treatment (FIG. 2H). Restoration of skin properties using the biophotonic treatment was as good as or better than treatment with silicone sheets.

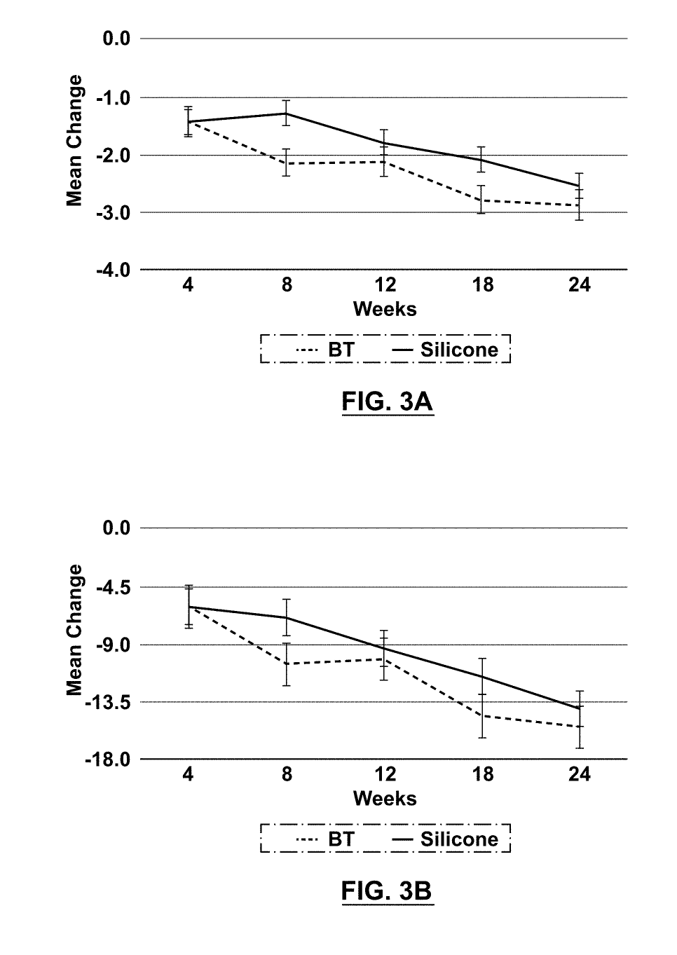

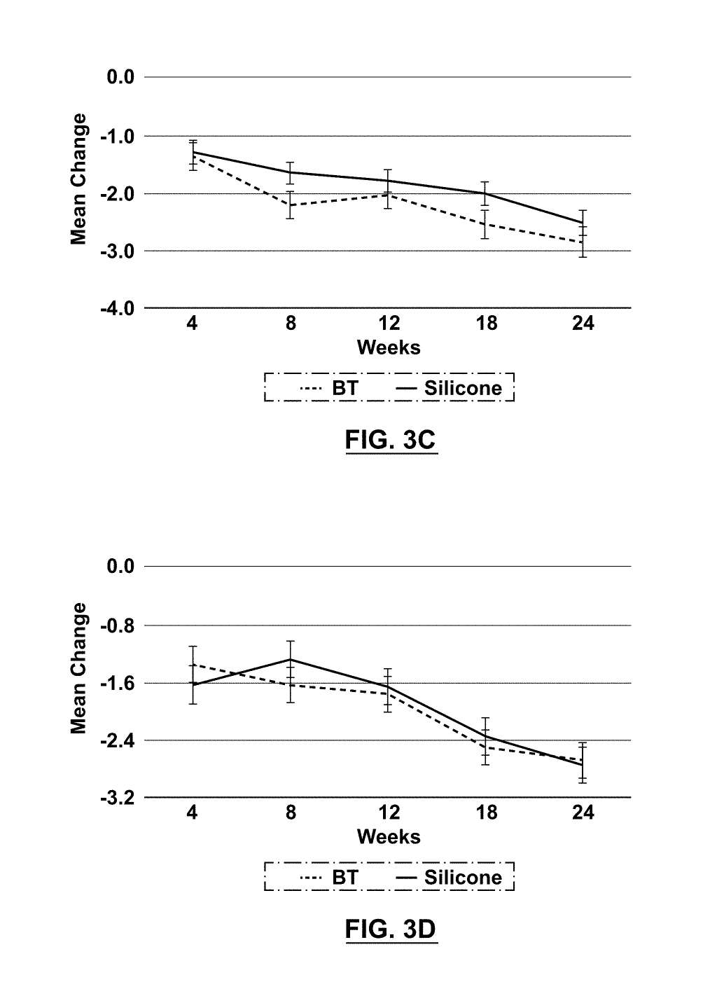

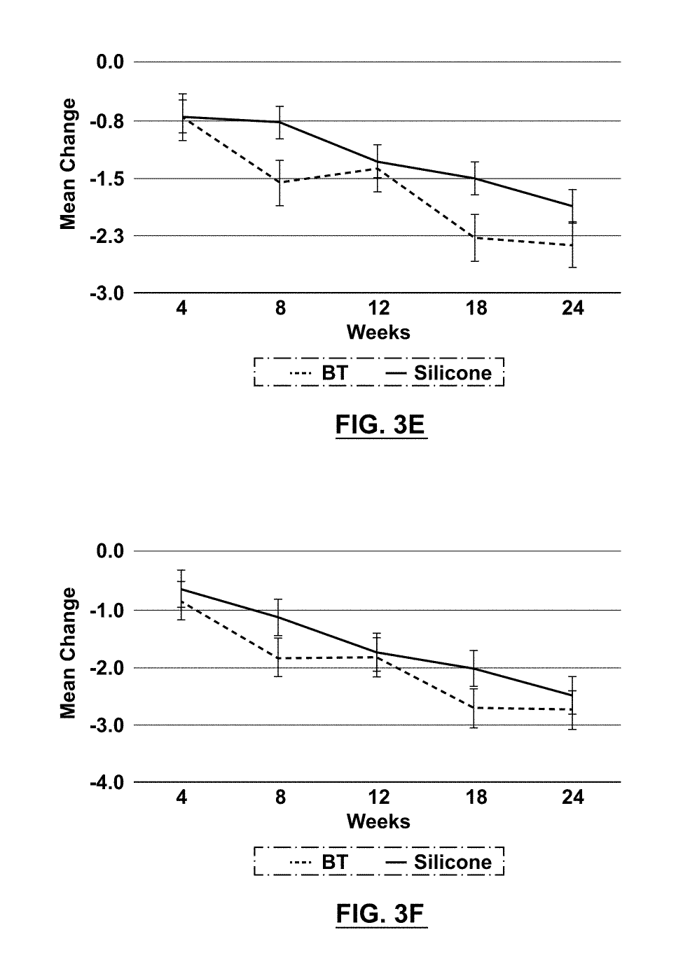

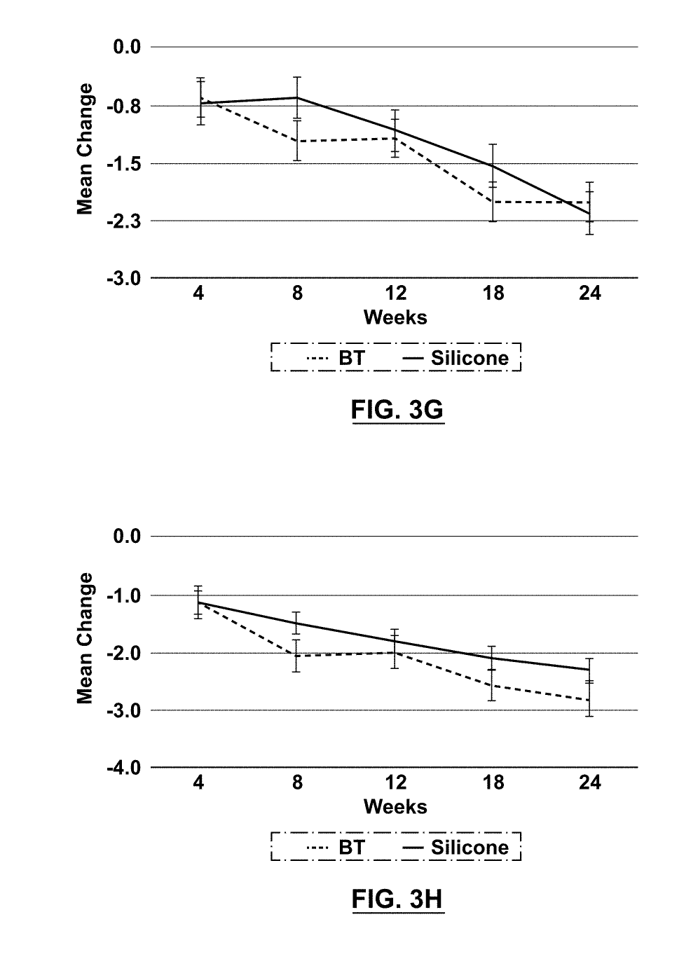

Patients in Group C1 and Group C2 showed reduced scarring of the acute wound at week 4 post-surgery compared to week 0 (baseline) (FIG. 3A and FIG. 3B), indicating that a double biophotonic treatment once a week for a period of about 4 weeks is efficient to reduce scarring. At 4 weeks, patients in Group C1 and Group C2 observed improved restoration of healthy skin properties such as, vascularity (FIG. 3C), pigmentation (FIG. 3D), skin thickness (FIG. 3E), pliability (FIG. 3F) and surface area (FIG. 3G) compared to baseline. Relief was also observed in patients treated with the biophotonic treatment (FIG. 3H). Restoration of skin properties using the biophotonic treatment was as good as or better than treatment with silicone sheets.

FIGS. 5A-5C show the breast of a subject in Group C1. The right breast (left panel) of the subject was treated according to Group C1 regimen, whereas the left breast (right panel) was treated with silicone sheets. FIG. 5A shows the breasts at week 7 post surgery; FIG. 5B shows the breasts at week 12 post surgery; FIG. 5C shows the breasts at week 24 post surgery. These photographs demonstrate that as early as week 7 post surgery, the double biophotonic treatment once weekly was more efficient in reducing scarring of the wound than treatment with the silicone sheets. The photographs further demonstrate that the double biophotonic treatment once weekly reduces surface area of the wound, increases vascularization of the wound, and helped in restoring pigmentation of the wound. The double biophotonic treatment once weekly was thus more efficient as reducing scarring of the wound than the single biophotonic treatment once or twice weekly.

Tables 5, 6, 7, and 8 below further outline the scoring obtained on the Vancouver Scar Scale for Groups A1, B1, A2, B2, C1 and C2 at visit #1 and at visit #26 on scar properties such as height (Table 5), pigmentation (Table 6), pliability (Table 7), and vascularity (Table 8). The scoring obtained indicate that the biophotonic treatment was efficient in improving these properties of the scar and was as efficient as or better than the silicone sheets in doing so. In particular, a lower proportion of patients treated with the biophotonic treatment showed red scars at visit #16 (week 24) than patients treated with silicone sheets. In addition, patients treated with the biophotonic treatment achieved normal scar height and normal pliability at visit #26 (week 24) than patients treated with silicone sheets.

TABLE 5
Assessment of height of the wound on the Vancouver Scar Scale

| | | | | NORMAL- | Grand |
|---|---|---|---|---|---|
| Row Labels | <2 MM | >5 MM | 2-5 MM | FLAT | Total |
| VISIT #1 | | | | | |
| Silicone | | | | | |
| A1 | 2 | | 4 | 1 | 7 |
| A2 | 1 | 1 | 3 | 2 | 7 |
| B1 | 2 | | 5 | | 7 |
| B2 | 4 | | 3 | | 7 |
| C1 | 3 | | 3 | 1 | 7 |
| C2 | 2 | | 4 | 1 | 7 |
| Silicone Total | 14 | 1 | 22 | 5 | 42 |
| BT | | | | | |
| A1 | | | 5 | 2 | 7 |
| A2 | 1 | | 4 | 2 | 7 |
| B1 | 2 | | 5 | | 7 |
| B2 | 4 | | 3 | | 7 |
| C1 | 3 | | 3 | 1 | 7 |
| C2 | 1 | | 5 | 1 | 7 |
| BT Total | 11 | | 25 | 6 | 42 |
| VISIT #26 | | | | | |
| Silicone | | | | | |
| A1 | 3 | | | 3 | 6 |
| A2 | 4 | | 1 | 1 | 6 |
| B1 | 3 | | | 2 | 5 |
| B2 | 4 | | | 1 | 5 |
| C1 | 4 | | 1 | | 5 |
| C2 | 4 | | | 3 | 7 |
| Silicone Total | 22 | | 2 | 10 | 34 |
| BT | | | | | |
| A1 | 2 | | 1 | 3 | 6 |
| A2 | 3 | | | 3 | 6 |
| B1 | | | | 5 | 5 |
| B2 | 2 | | | 3 | 5 |
| C1 | 2 | | 1 | 2 | 5 |
| C2 | 2 | | | 5 | 7 |
| BT Total | 11 | | 2 | 21 | 34 |

TABLE 6
Assessment of pigmentation of the
wound on the Vancouver Scar Scale

| | HYPER- | HYPO- | | |
|---|---|---|---|---|
| | PIGMENTA- | PIGMENTA- | | Grand |
| Row Labels | TION | TION | NORMAL | Total |
| VISIT #1 | | | | |
| Silicone | | | | |
| A1 | 4 | | 3 | 7 |
| A2 | 4 | | 3 | 7 |
| B1 | 3 | | 4 | 7 |
| B2 | 4 | | 3 | 7 |
| C1 | 5 | | 2 | 7 |
| C2 | 5 | 1 | 1 | 7 |
| Silicone Total | 25 | 1 | 16 | 42 |
| BT | | | | |
| A1 | 4 | | 3 | 7 |
| A2 | 4 | | 3 | 7 |
| B1 | 3 | | 4 | 7 |
| B2 | 4 | | 3 | 7 |
| C1 | 5 | | 2 | 7 |
| C2 | 6 | | 1 | 7 |
| BT Total | 26 | | 16 | 42 |
| VISIT #26 | | | | |
| Silicone | | | | |
| A1 | 1 | 2 | 3 | 6 |
| A2 | 2 | 2 | 2 | 6 |
| B1 | 3 | | 2 | 5 |
| B2 | | 1 | 4 | 5 |
| C1 | | | 5 | 5 |
| C2 | 3 | | 4 | 7 |
| Silicone Total | 9 | 5 | 20 | 34 |
| BT | | | | |
| A1 | 2 | 2 | 2 | 6 |
| A2 | 1 | 2 | 3 | 6 |
| B1 | 2 | | 3 | 5 |
| B2 | 3 | 1 | 1 | 5 |
| C1 | 1 | | 4 | 5 |
| C2 | 2 | | 5 | 7 |
| BT Total | 11 | 5 | 18 | 34 |

TABLE 7
Assessment of pliability of the wound on the Vancouver Scar Scale

| | BAND- | | NOR- | | YIELD- | Grand |
|---|---|---|---|---|---|---|
| Row Labels | ING | FIRM | MAL | SUPPLE | ING | Total |
| VISIT #1 | | | | | | |
| Silicone | | | | | | |
| A1 | 1 | 3 | | 2 | 1 | 7 |
| A2 | | 4 | 1 | 1 | 1 | 7 |
| B1 | | 7 | | | | 7 |
| B2 | | 3 | | 1 | 3 | 7 |
| C1 | | 5 | | 1 | 1 | 7 |
| C2 | | 6 | | | 1 | 7 |
| Silicone Total | 1 | 28 | 1 | 5 | 7 | 42 |
| BT | | | | | | |
| A1 | 1 | 3 | | 2 | 1 | 7 |
| A2 | | 5 | 1 | 1 | | 7 |
| B1 | | 7 | | | | 7 |
| B2 | | 5 | | 1 | 1 | 7 |
| C1 | | 4 | | | 3 | 7 |
| C2 | | 7 | | | | 7 |
| BT Total | 1 | 31 | 1 | 4 | 5 | 42 |
| VISIT #26 | | | | | | |
| Silicone | | | | | | |
| A1 | | | 2 | 3 | 1 | 6 |
| A2 | | | 1 | 3 | 2 | 6 |
| B1 | | | 2 | 2 | 1 | 5 |
| B2 | | | 1 | 3 | 1 | 5 |
| C1 | | 1 | | 3 | 1 | 5 |
| C2 | | | 2 | 4 | 1 | 7 |
| Silicone Total | | 1 | 8 | 18 | 7 | 34 |
| BT | | | | | | |
| A1 | | | 2 | 3 | 1 | 6 |
| A2 | | | 2 | 4 | | 6 |
| B1 | | | 2 | 3 | | 5 |
| B2 | | | 1 | 4 | | 5 |
| C1 | | 1 | 1 | 2 | 1 | 5 |
| C2 | | 1 | 5 | | 1 | 7 |
| BT Total | | 2 | 13 | 16 | 3 | 34 |

TABLE 8
Assessment of the vascularity of the
wound on the Vancouver Scar Scale

| | | | | | Grand |
|---|---|---|---|---|---|
| Row Labels | NORMAL | PINK | PURPLE | RED | Total |
| VISIT #1 | | | | | |
| Silicone | | | | | |
| A1 | | 1 | | 6 | 7 |
| A2 | | 1 | 2 | 4 | 7 |
| B1 | | 1 | | 6 | 7 |
| B2 | | 2 | 1 | 4 | 7 |
| C1 | | 2 | 1 | 4 | 7 |
| C2 | | 1 | 1 | 5 | 7 |
| Silicone Total | | 8 | 5 | 29 | 42 |
| BT | | | | | |
| A1 | | 1 | | 6 | 7 |
| A2 | 1 | | 2 | 4 | 7 |
| B1 | | 1 | | 6 | 7 |
| B2 | | 2 | 1 | 4 | 7 |
| C1 | | 3 | 1 | 3 | 7 |
| C2 | | | 1 | 6 | 7 |
| BT Total | 1 | 7 | 5 | 29 | 42 |
| VISIT #26 | | | | | |
| Silicone | | | | | |
| A1 | 1 | 4 | | 1 | 6 |
| A2 | 1 | 3 | | 2 | 6 |
| B1 | 1 | 3 | | 1 | 5 |
| B2 | | 5 | | | 5 |
| C1 | 1 | 3 | | 1 | 5 |
| C2 | 2 | 4 | | 1 | 7 |
| Silicone Total | 6 | 22 | 0 | 6 | 34 |
| BT | | | | | |
| A1 | 1 | 4 | | 1 | 6 |
| A2 | 2 | 4 | | | 6 |
| B1 | 4 | 1 | | | 5 |
| B2 | 2 | 1 | | 2 | 5 |
| C1 | 1 | 4 | | | 5 |
| C2 | 4 | 2 | | 1 | 7 |
| BT Total | 14 | 16 | 0 | 4 | 34 |

TABLE 9
Avg. results of POSAS Observer Scale (visit #26)

| | BT treated | Silicone sheet |
|---|---|---|
| Properties | breast | treated breast |
| Vascularity | 1.89 | 2.40 |
| Pigmentation | 1.92 | 2.07 |
| Thickness | 2.07 | 2.28 |
| Relief | 1.78 | 2.23 |
| Pliability | 1.85 | 2.19 |
| Surface Area | 2.02 | 2.21 |
| Overall Opinion | 1.89 | 2.26 |
| Color | 4.57 | 5.0 |
| Stiffness | 3.85 | 4.54 |
| Irregularity | 4.23 | 4.83 |

The single biophotonic treatment once or twice weekly and the double biophotonic treatment once weekly reduce the scarring process and improve the overall healing of the wound. The results obtained further demonstrate that the biophotonic treatment of the present disclosure is as good as or better results at reducing scarring of the wound than treatment with silicone sheets.

It should be appreciated that the invention is not limited to the particular embodiments described and illustrated herein but includes all modifications and variations falling within the scope of the technology as defined in the appended claims.

All documents referred to herein are hereby incorporated by reference into the present application.

1. (canceled)

2. A method for reducing scarring of a wound, comprising:
a) at least once weekly, performing an application step followed by an illumination step, wherein the application step comprises topically applying a biophotonic composition on the wound, and wherein the illumination step comprises illuminating the applied biophotonic composition with actinic light for at least 5 minutes;
b) allowing a rest period of less than about one week after the illumination step; and
c) repeating a) and b) over a period of at least about 4 weeks.

3. The method as defined in claim 2, wherein a) and b) are performed at least twice weekly.

4. The method as defined in claim 3, wherein a first occurrence and a second occurrence of the method are performed consecutively.

5. The method as defined in claim 4, wherein the first occurrence and the second occurrence are performed once weekly.

6. The method as defined in claim 2, wherein a) and b) are performed at least three times a week.

7.-10. (canceled)

11. The method as defined in claim 2, wherein the method is performed over a period of between about 4 weeks to about 24 weeks.

12. The method as defined in claim 2, wherein the rest period is of about 3 days.

13. The method as defined in claim 2, further comprising a step between step b) and step c), wherein the step between step b) and step c) includes removing the biophotonic composition from the wound after step b).

14. The method as defined in claim 2, wherein the wound is at least one of an acute wound, a surgical wound, and a traumatic wound.

15.-16. (canceled)

17. The method as defined in claim 2, further comprising at least one of improving vascularity of the wound, improving pigmentation of the wound, decreasing thickness of the wound, and reducing surface area of the wound.

18.-20. (canceled)

21. The method as defined in claim 2, wherein the biophotonic composition comprises at least one light-accepting molecule.

22.-23. (canceled)

24. The method as defined in claim 21, wherein the at least one light-accepting molecule is selected from Eosin Y, Eosin B, Erythrosin B, Fluorescein, Rose Bengal and Phloxin B.

25. The method as defined in claim 24, wherein the at least one light-accepting molecule is Eosin Y.

26. The method as defined in claim 21, wherein the at least one light-accepting molecule is present in an amount of between about 0.001% to about 40% by weight of the total biophotonic composition.

27.-28. (canceled)

29. The method as defined in claim 2, wherein the biophotonic composition is illuminated for a period of between about 5 minutes to about 30 minutes.

30.-31. (canceled)

32. The method as defined in claim 29, wherein the biophotonic composition is illuminated for less than about 10 minutes.

33.-34. (canceled)

35. The method as defined in claim 2, wherein the biophotonic composition is illuminated with violet or blue light or both.

36. The method as defined in claim 2, wherein the biophotonic composition is illuminated with light having a power density of less than about 150 mW/cm².

37. (canceled)

38. The method as defined in claim 2, wherein the method is performed on a closed acute wound.

39. (canceled)

40. A method for reducing scarring of a wound, wherein the method comprises topically applying on the wound a biophotonic composition followed by illumination of the applied biophotonic composition with actinic light, wherein the method comprises the following schedule:
(a) at least once weekly:
i) topically applying a first amount of biophotonic composition on the wound, illuminating the applied first amount of biophotonic composition for a period of at least 5 minutes, removing the applied first amount of biophotonic composition from the wound; and
ii) topically applying a second amount of biophotonic composition on the wound, illuminating the applied second amount of biophotonic composition for a period of at least 5 minutes, removing the applied second amount of biophotonic composition from the wound; and
iii) initiating a rest period of less than about a week; and
(b) repeating step (a) over a period of at least 4 weeks.

41.-54. (canceled)
